(19) United States
(12) Patent Application Publication
(10) Pub. No.: US 2005/0203013 A1
(43) Pub. Date: Sep. 15, 2005

(54) Methods for inhibiting vascular permeability

(76) Inventors: Shay Soker, Greensboro, NC (US); Ronit Satchi-Fainaro, Chestnut Hill, MA (US)

Correspondence Address: David S. Resnick;NIXON PEABODY LLP, 100 Summer Street, Boston, MA, 02110-2131, US

(21) Appl. No.: 10/962,723

(22) Filed: Oct. 12, 2004

Related U.S. Application Data
(63) Continuation-in-part of application No. PCT/US03/11265, filed on Apr. 11, 2003.
(60) Provisional application No. 60/371,841, filed on Apr. 11, 2002.

(51) Int. Cl. A61K 38/17; A61K 38/54

(52) U.S. Cl. 514/12

(57) ABSTRACT

The present invention relates to methods for decreasing or inhibiting disorders associated with vascular hyperpermeability and to methods of screening for compounds that affect permeability, angiogenesis and stabilize tight junctions. In one aspect of the present invention there is provided a method of decreasing or inhibiting vascular hyperpermeability in an individual in need of such treatment. The method includes administering to the individual an effective amount of an antiangiogenic compound selected from the group consisting of endostatin, thrombospondin, angiostatin, tumstatin, arrestin, recombinant EPO and polymer conjugated TNP-470. Other antiangiogenic compounds are disclosed herein.

### CROSS-REFERENCE TO RELATED APPLICATIONS

This application is a Continuation-In-Part of International Application PCT/US2003/011265, filed Apr. 11, 2003, which claims the benefit under 35 U.S.C § 119(e) of U.S. Provisional Application 60/371,841, filed Apr. 11, 2002.

### GOVERNMENT FUNDING

This invention was made with government support under P01 CA45548, R01 CA064481, and R01 CA37395 awarded by the National Institutes of Health. The government has certain rights in the invention.

### FIELD OF THE INVENTION

The present invention relates to methods for decreasing or inhibiting disorders associated with vascular hyperpermeability and to methods of screening for compounds that affect permeability, angiogenesis and stabilize tight junctions.

### BACKGROUND OF THE INVENTION

Vascular hyperpermeability has been implicated in numerous pathologies including vascular complications of diabetes, pulmonary hypertension and various edemas, and has been rendered responsible for decreasing efficacy of anti-cancer therapies due to loss of endogenous angiogenesis inhibitors into the urine. For instance, a complication of diabetes, diabetic retinopathy is a leading cause of blindness that affects approximately 25% of the estimated 16 million Americans with diabetes. It is believed that diabetic retinopathy is induced by hypoxia in the retina as a result of hyperglycemia.

The degree of diabetic retinopathy is highly correlated with the duration of diabetes. There are two kinds of diabetic retinopathy. The first, non-proliferative retinopathy, is the earlier stage of the disease characterized by increased capillary permeability, microaneurysms, hemorrhages, exudates, and edema. Most visual loss during this stage is due to the fluid accumulating in the macula, the central area of the retina. This accumulation of fluid is called macular edema, and can cause temporary or permanent decreased vision. The second category of diabetic retinopathy is called proliferative retinopathy and is characterized by abnormal new vessel formation, which grows on the vitreous surface or extends into the vitreous cavity. Neovascularization can be very damaging because it can cause bleeding in the eye, retinal scar tissue, diabetic retinal detachments, or glaucoma, any of which can cause decreased vision or blindness.

Current treatment of non-proliferative retinopathy includes intensive insulin therapy to achieve normal glycemic levels in order to delay further progression of the disease, whereas the current treatment of proliferative retinopathy involves panretinal photocoagulation and vitrectomy. The treatment of non-proliferative retinopathy, while valid in theory, is mostly ineffective in practice because it usually requires considerable modification in the lifestyle of the patients, and many patients find it very difficult to maintain the near-normal glycemic levels for a time sufficient to slow and reverse the progression of the disease. Thus, the current treatment of non-proliferative retinopathy only delays the progression of the disease and cannot be applied effectively to all patients who require it.

Another complication of diabetes, diabetic nephropathy is the dysfunction of the kidneys and the most common cause of end-stage renal disease in the USA. It is a vascular complication that affects the glomerular capillaries of the kidney and reduces the kidney's filtration ability. Nephropathy is first indicated by the appearance of hyperfiltration and then microalbuminuria. Heavy proteinuria and a progressive decline in renal function precede end-stage renal disease. It is believed that hyperglycemia causes glycosylation of glomerular proteins, which may be responsible for mesangial cell proliferation and matrix expansion and vascular endothelial damage. Typically before any signs of nephropathy appear, retinopathy has usually been diagnosed.

Early treatment of nephropathy can attenuate disease progression. Currently, aggressive treatment is indicated including protein, sodium and phosphorus restriction diet, intensive glycemic control, ACE inhibitors (e.g., captopril) and/or nondihydropyridine calcium channel blockers (diltiazem and verapamil), C-peptide and somatostatin are also used. The treatment regimen for early-stage nephropathy comprising dietary and glycemic restrictions is less effective in practice than in theory due to difficulties associated with patient compliance. Renal transplant is usually recommended to patients with end-stage renal disease due to diabetes. Survival rate at 5 years for patients receiving a transplant is about 60% compared with only 2% for those on dialysis. Renal allograft survival rate is greater than 85% at 2 years.

Vascular hyperpermeability plays an important role in complications of nephrotic syndrome. Nephrotic syndrome is a condition characterized by massive edema (fluid accumulation), heavy proteinuria (protein in the urine), hypoalbuminemia (low levels of protein in the blood), and susceptibility to infections. Nephrotic syndrome results from damage to the kidney's glomeruli. Glomeruli are tiny blood vessels that filter waste and excess water from the blood. The damaged glomeruli are characterized by hyperpermeability. Nephrotic syndrome can be caused by glomerulonephritis, diabetes mellitus, or amyloidosis. Presently, prevention of nephrotic syndrome relies on controlling these diseases.

One serious complication of nephrotic syndrome is thrombosis (blood clotting), especially in the brain. The loss of plasma proteins due to hyperpermeability of the glomeruli in patients with nephrotic syndrome leads to a reduced concentration of Antithrombin III (ATIII). ATIII is one of the most important regulators of the coagulation system. Low levels of ATIII in the blood means a great and well established risk for thrombotic complications, especially blood clots in the brain. Decreasing permeability of glomeruli would prevent thrombosis.

Vascular hyperpermeability has also been found to play a role in pathophysiology of nephrotic edema in human primary glomerulonephritis, such as idiopathic nephrotic syndrome (INS). It is believed that vascular hyperpermeability in nephrotic edema is related to the release of vascular permeability factor and other cytokines by immune cells. See Rostoker et al., Nephron 85:194-200 (2000).

Pulmonary hypertension is a rare blood vessel disorder of the lung in which the pressure in the pulmonary artery (the blood vessel that leads from the heart to the lungs) rises above normal levels and may become life threatening. Pulmonary hypertension has been historically chronic and incurable with a poor survival rate. Recent data indicate that the length of survival is continuing to improve, with some patients able to manage the disorder for 15 to 20 years or longer.

Pulmonary hypertension is caused by alveolar hypoxia, which results from localized inadequate ventilation of well-perfused alveoli or from a generalized decrease in alveolar ventilation. Treatment of pulmonary hypertension usually involves continuous use of oxygen. Pulmonary vasodilators (e.g., hydralazine, calcium blockers, nitrous oxide, prostacyclin) have not proven effective. Lung transplant is typically recommended to patients who do not respond to therapy.

It is well known that the members of the vascular endothelial growth factor (VEGF) family induce vascular permeability. Compounds designed to inhibit the activity of VEGF, including anti-VEGF antibodies, anti-VEGF receptor antagonists and small molecules that inhibit receptor tyrosin kinase, activity should also inhibit VEGF induced vascular permeability. However, these compounds would have no effect on vascular permeability that is VEGF-independent. It would be desirable to have a method to inhibit both VEGF-independent and dependent vascular permeability and thus provide alternatives to treating disorders whose pathology is associated with vascular hyperpermeability, such as non-proliferative diabetic retinopathy, diabetic nephropathy, nephrotic syndrome, pulmonary hypertension and various edemas.

### SUMMARY OF THE INVENTION

In one aspect of the present invention there is provided a method of decreasing or inhibiting vascular hyperpermeability in an individual in need of such treatment. The method includes administering to the individual an effective amount of an antiangiogenic compound selected from the group consisting of endostatin, thrombospondin, angiostatin, tumstatin, arrestin, recombinant EPO and polymer conjugated TNP-470. Other antiangiogenic compounds are disclosed herein.

An “antiangiogenic compound”, as used herein, is a compound capable of inhibiting the formation of blood vessels. The disease associated with vascular permeability for treatment with the present invention includes vascular complications of diabetes such as non-proliferative diabetic retinopathy and diabetic nephropathy; nephrotic syndrome; pulmonary hypertension; burn edema; tumor edema; brain tumor edema; IL-2 therapy-associated edema; “Reperfusion” syndromes following ischemic injury in brain and heart, transplantation of organs, and surgery for removal of large tumors in the pelvis where major vessels must be occluded temporarily; Cerebral edema associated with brain tumors, head injury or stroke; Lymphedema associated with axillary lymph node dissection following mastectomy; and Allergic reactions associated with edema.

The method of the invention can be used to prevent the leakage from blood vessels of natural angiogenesis inhibitors.

In yet another aspect of the present invention there is provided a method of treating and/or preventing a disease associated with vascular hyperpermeability in an individual in need of such treatment. The method involves administering to the individual an effective amount of a compound capable of increasing cell-cell contacts by stabilizing tight junction complexes and increasing contact with the basement membrane. Effective compounds are, for example, endostatin, thrombospondin, angiostatin, tumstatin, arrestin, recombinant EPO and polymer conjugated TNP-470. In certain embodiments, it may be desirable to conjugate the antiangiogenic agent with a polymer. An HPMA copolymer is preferred.

In a further aspect of the invention there is provided a method of screening for compounds that stabilize tight junction complexes. The method involves culturing endothelial cells in the presence of a test compound, incubating with the cultured endothelial cells expressing junction proteins, and assessing whether the test compound stabilized the tight junction complexes. The assessment of stabilization of a tight junction protein can be readily performed by immunostaining for that protein and visualized under fluorescent microscopy. Intense cell-boundary staining is indicative of a compound that stabilizes the tight junction protein, and, therefore, is indicative of an anti-permeability and/or an anti-angiogenic activity which can be further tested for such activity. The tight junction proteins contemplated by the present invention include integral membrane proteins, cytoplasmic proteins, and proteins associated with tight junctions. More particularly, the tight junction proteins include occludin, claudin, zonula occludens (ZO)-1, -2, -3, catenins, VE cadherin, cingulin and p130.

In a further aspect of the invention there is provided a method of screening for compounds that affect vascular permeability. The method involves assaying endothelial cells on a permeable substrate (e.g., a collagen coated inserts of “Transwells”), contacting the assay with a test compound, treating the assay with a mixture of markers (e.g., FITC label) and permeability-inducing agents (e.g., vascular endothelial growth factor (VEGF) and platelet-activating factor (PAF) among others), and measuring the amount of marker to travel through the substrate. The test compound with antipermeability properties would cause the marker to diffuse slower compare to the control and to permeability-inducing agents.

In another aspect of the present invention there is provided a method for assessing bioeffectiveness of an antiangiogenic compound in a patient being treated with such compound. The method involves administering to the patient an intradermal/subcutaneous injection of histamine before treating the patient with the antiangiogenic compound and measuring a histamine-induced local edema. Thereafter, treating the patient with the antiangiogenic compound, and again administering to said patient an intradermal/subcutaneous injection of histamine subsequent to treating the patient with the antiangiogenic compound and measuring the histamine-induced local edema. A decrease in the measurement of the histamine-induced local edema compared to that seen before the treatment with the antiangiogenic compound indicates that the compound is bioeffective.

The present invention also provides an alternative method for assessing bioeffectiveness of an antiangiogenic compound in a patient being treated with such compound. The method involves measuring a level of a protein in a bodily fluid of the patient (e.g., blood or urine) before treating the patient with the antiangiogenic compound, then, treating the patient with the antiangiogenic compound and measuring the level of the protein in the bodily fluid of the patient. A decrease in the level of the protein in the bodily fluid compare to the pre-treatment level indicates that the compound inhibits vascular permeability and is bioeffective.

Finally, the present invention provides an article of manufacture which includes packaging material and a pharmaceutical agent contained within the packaging material. The packaging material includes a label which indicates said pharmaceutical may be administered, for a sufficient term at an effective dose, for treating and/or preventing a disease associated with vascular permeability. The pharmaceutical agent is selected from the group consisting of endostatin, thrombospondin, angiostatin, tumstatin, arrestin, recombinant EPO and polymer conjugated TNP-470. The disease associated with vascular permeability includes, but not limited to, vascular complications of diabetes such as non-proliferative diabetic retinopathy and diabetic nephropathy, nephrotic syndrome, pulmonary hypertension, burn edema, tumor edema, brain tumor edema, IL-2 therapy-associated edema, and other edema-associated diseases.

Unless otherwise defined, all technical and scientific terms used herein have the same meaning as commonly understood by one of ordinary skill in the art. Although methods and materials similar or equivalent to those described herein can be used in the practice or testing of the invention, the preferred methods and materials are described below. All publications, patent applications, patents and other references mentioned herein are incorporated by reference. In addition, the materials, methods and examples are illustrative only and not intended to be limiting. In case of conflict, the present specification, including definitions, controls.

### BRIEF DESCRIPTION OF THE DRAWINGS

The accompanying drawings, which are incorporated in and constitute a part of this specification, illustrate embodiments of the invention and, together with the description, serve to explain the objects, advantages, and principles of the invention.

FIGS. 1A to 1B show a quantitative analysis of Evans Blue dye extravasation showing lower skin capillary permeability of the antiangiogenic factor-treated mice and indicate the weak permeability-inducing effect of VEGF in these mice. FIG. 1A, O.D. at 620 nm. FIG. 1B, O.D. as a % of PBS treated mice.

FIGS. 2A to 2B show a quantitative analysis of Evans Blue dye extravasation showing lower skin capillary permeability of the endostatin-treated mice compared with control and the lack of PAF-induced hyperpermeability in these mice. FIG. 2A, O.D. at 620 nm. FIG. 2B, O.D. as a % of saline treated mice.

FIG. 3 is a quantitative analysis of skin vessel permeability of saline and endostatin-treated mice, during a contiguous period of time, and skin vessel permeability in response to PAF injection.

FIG. 4 illustrates that endostatin treatment significantly reduces the diffusion of large molecules through the endothelial cell monolayer.

FIGS. 5 and 6 show kinetics of the diffusion process using 10 kDa dextran (FIG. 5) and 70 kDa dextran (FIG. 6).

FIGS. 7A-7E show that free and polymer conjugated TNP-470 prevents VEGF, PAF and histamine-induced vascular leakage compare to control in the miles assay.

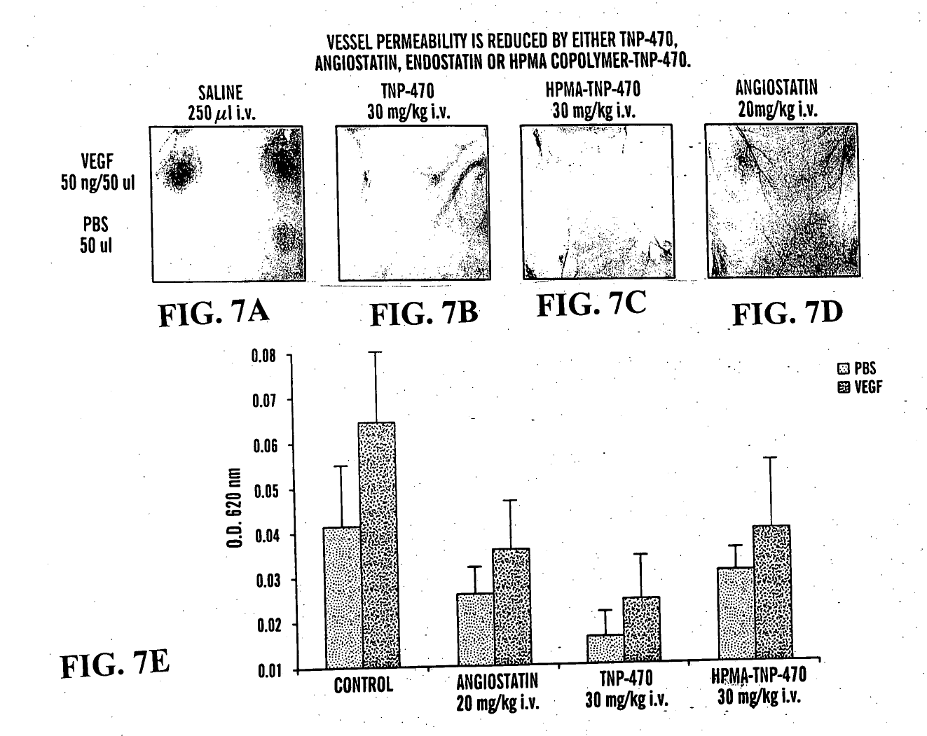

FIGS. 8A-8D show that the “indirect” angiogenesis inhibitors, Thalidomide and Herceptin, have no effect on vessel permeability.

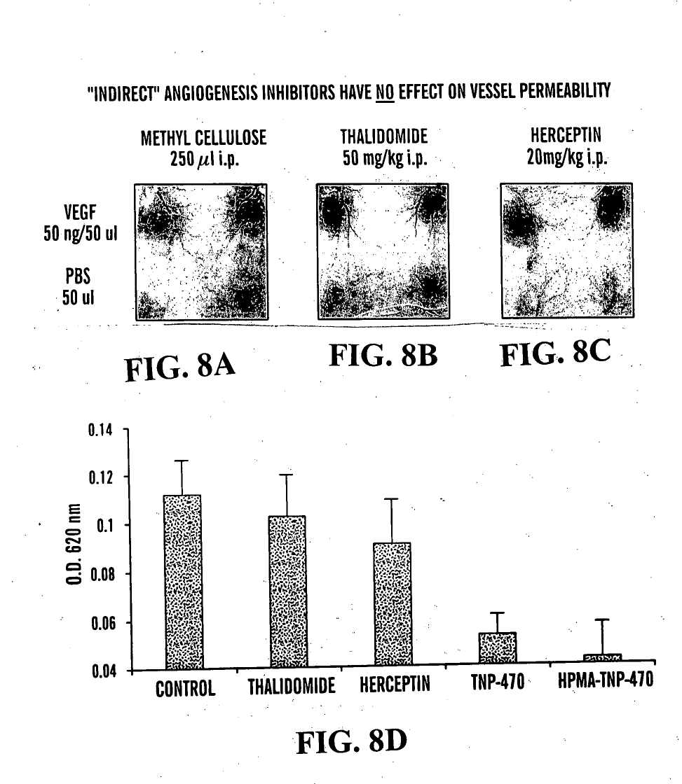

FIG. 9 shows the permeability effects in SCID mice bearing A2058 human melanoma treated for 3-5 days with angiostatin, TNP-470 and polymer conjugated TNP-470 prior to the Miles assay.

FIG. 10 shows bovine capillary endothelial (BCE) cells treated with TNP-470 for 3 days and stained with antibody to the tight junction protein ZO-1.

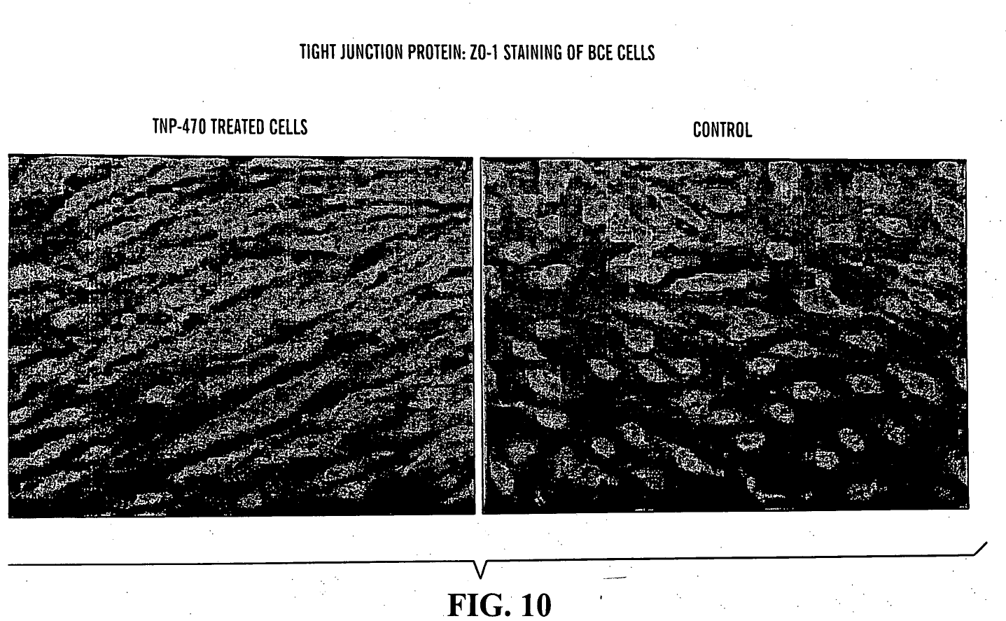

FIG. 11 shows the relative weight of the lungs following treatment with TNP-470 for 3 days compared to control lungs after induction of edema with IL-2 i.m. administration and control normal lungs. As shown in the graph, TNP-470 reduces pulmonary edema.

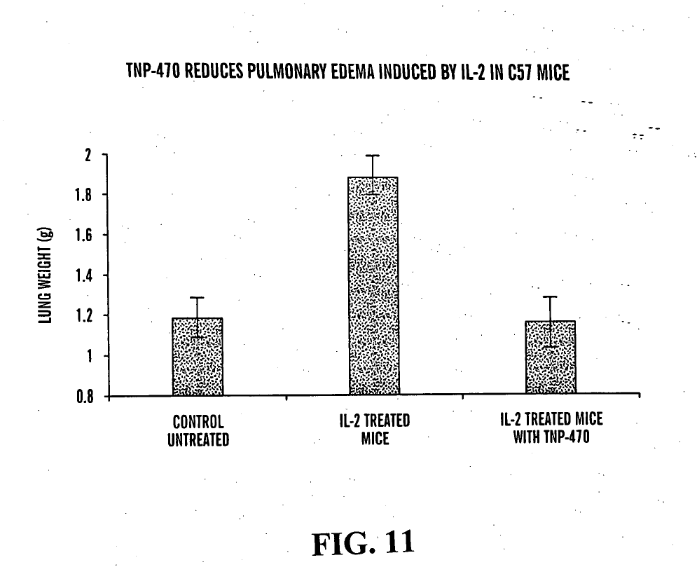

FIG. 12 shows the results in the Miles assay in SCID mice bearing A 2058 human melanoma treated for 5 days with endostatin.

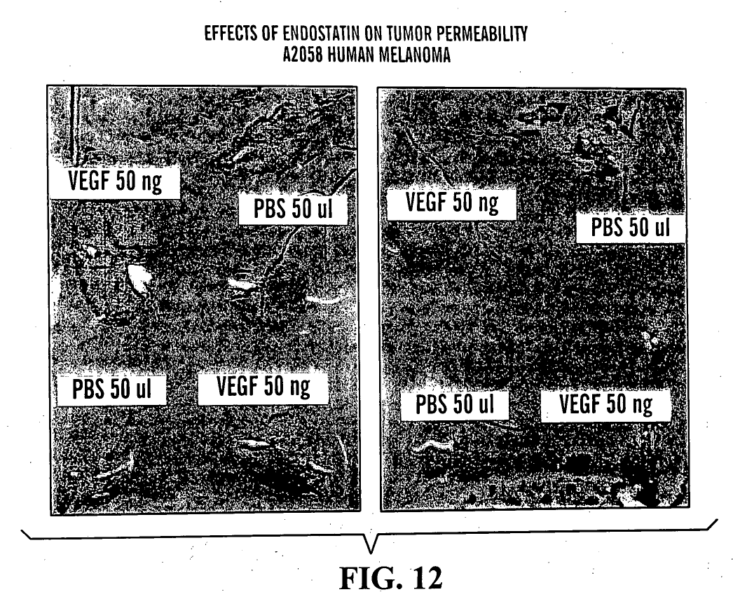

FIGS. 13A-13C show that TNP-470 prevents vascular permeability in mouse skin capillaries in the Miles vascular permeability assay. (FIG. 13A) The inner dorsal skin of pretreated SCID mice injected locally with PBS or VPF/VEGF was exposed. Faint blue color (not shown) in free or conjugated TNP-470 and angiostatin than corresponding treatment with thalidomide, herceptin or methyl cellulose and saline (as controls). (FIG. 13B) The blue areas of skin were excised and extracted dye contents were quantified by spectrophotometry at 620 nm. Data are expressed as mean ±S.E. TNP-470 and HPMA-TNP-470. (FIG. 13C) quantification of dye extracted from PBS (black columns), VEGF (gray columns), PAF (white columns) and histamine (striped columns)-induced permeability sites following treatment of mice with TNP470, P-TNP-470 conjugate, agiostatin or saline. Data are expressed as mean±S.E.

FIGS. 14A-14C show that TNP-470 decreases ear swelling in DTH reactions elicited by oxazolone. DTH reactions were induced in the ear skin of C57B1/6J mice using oxazolone challenge. (FIG. 14A) Ear swelling is expressed as the increase (Δμm) over the original ear thickness in micrometers. Mice treated with TNP-470 (squares) showed a significantly decreased ear swelling (P<0.01) 24 hours after challenge as compared with saline-injected challenged mice (circles). Control left ears treated with vehicle alone in both groups showed no swelling (diamonds and triangles). (FIG. 14B) Macroscopically visible increase of ear swelling and erythema in control mice (left panel) as compared with TNP-470-treated mice (right panel) at 24 hours after oxazolone challenge. (FIG. 14C) H&E staining shows increased extravasation of infiltrate into the extracellular matrix in control mice compared to TNP-470-treated mice and arrows mark lymphatics (see arrows).

FIGS. 15A-15B show that TNP-470 prevents IL-2-induced pulmonary edema. (FIG. 15A) Mice were pretreated with saline or TNP-470 for 3 days and then injected with IL-2 for 5 days. Mice were euthanized and lungs were dissected and weighed. FIG. 15B, Histological examnation of lungs of IL-2 treated mice +/−TNP40.

FIGS. 16A-16C show that TNP-470 reduces tumor blood vessel permeability. (FIG. 16A) VEGF levels in conditioned media of several cell lines measured by ELISA. The growth of all tumors tested for permeability in (FIG. 16B) is known from the literature to be inhibited by TNP-470 as shown in the right column of the table. (FIG. 16B) Mice bearing lewis lung carcinoma (LLC), A2058 melanoma, MCF-7 breast carcinoma, MDA-MB-231 breast carcinoma, BXPC3 pancreatic adenocarcinoma or U87 glioblastoma were treated with saline (100%), TNP-470 (black,columns), HPMA copolymer-TNP-470 (P-TNP-470) (gray columns) or angiostatin (white columns, only LLC, A2058 and U87) for 3-5 days. Following treatment Evan's blue dye was injected and after 10 minutes tumors were excised, weighed and the dye content per 100 mg tumor tissue was quantified spectrophotometrically at 620 nm. For each tumor, control group was determined as 100% permeability. (FIG. 16C) C57 mice were treated with TNP-470 (30 mg/kg/d s.c. for 3 days) or with saline. Then injected with 100 μl 1% Evan's Blue i.v. and after 10 min injected VEGF intradermally at different concentrations. Skin punch biopsies were collected and extracted dye in formamide was read at 620 nm. Control saline-treated mice showed a dose-response correlation between increasing VEGF injection and dye accumulation, up to saturation. TNP-470-treated mice showed inhibition of permeability up to 25 ng but, above that dose, TNP-470 lost its effectiveness in inhibiting permeability and dye accumulation.

FIGS. 17A-17H show that TNP-470 does not affect vesiculo-vacuolar organelle (VVO) or endothelial junction structures. Venule endothelial cells in mice injected systemically with buffer (FIG. 17A, FIG. 17B) or TNP-470 (FIG. 17C, FIG. 17D). Inter-endothelial cell junctions (FIG. 17A, FIG. 17C) are normally closed and VVOs are normal (FIG. 17B, FIG. 17D) in both sets of animals. There is minor leakage of intravenously injected circulating ferritin (FIG. 17B, small particles, some of which are in the lumen and the extravascular space) via VVOs (arrow marks a ferritin-containing vesicle). Leakage was reduced in the TNP-470 treated set (FIG. 17C, FIG. 17D). Venule endothelial cells in mice injected locally with VEGF and systemically with buffer (FIG. 17E, FIG. 17F) or with TNP-470 (FIG. 17G, FIG. 17H). In both sets of mice inter-endothelial cell junctions are normally closed (FIG. 17E, FIG. 17G) and VVOs are normal. Intravenously injected ferritin is seen to be extravasating through VVO vesicles (FIG. 17F, arrows) but to a lesser extent in TNP-470-treated mice (FIG. 17G, FIG. 17H). L, vascular lumen; p, pericyte. Bars:200 nm.

FIGS. 18A-18G show that free and conjugated TNP-470 inhibit VPF/VEGF-induced VEGFR-2 phosphorylation, endothelial cell proliferation, Ca influx and MAPK in vitro. (FIG. 18A) HMVEC-d and (FIG. 18B) HUVEC cells were incubated for 5 minutes with 10 ng/ml VPF/VEGF with or without TNP-470 and HPMA copolymer-TNP470 for 2 hours as follows: (1) control (no VPF/VEGF or drug), (2) VPF/VEGF alone, (3) TNP-470 alone, (4) VPF/VEGF, TNP-470, and for B HMVEC-d also (5) HPMA copolymer-TNP-470 alone, and (6) VPF/VEGF, HPMA copolymer-TNP-470. Cells were extracted and immunoprecipitated with an antibody to VEGFR-2. Immunoprecipitates (IP) were then captured with protein A-agarose beads. Beads were washed and IP solubilized by boiling in SDS-buffer and subjected to SDS-PAGE and Western blotting with an antibody to phosphotyrosine (pTyr). Blots were stripped and probed for VEGFR-2 to show equal loading. (FIG. 18C) TNP-470 inhibited U87 glioblastoma (▪) and HMVEC-d (●) proliferation after 72 hours. The solid line represents the proliferation of growth factor-induced cells (—) and the dotted line represents cell proliferation in the absence of growth factors ( - - - ). Decrease of (FIG. 18D) VEGF-, (FIG. 18E) histmanine-, and (FIG. 18F) PAF-induced-Ca²⁺ influx in HMVEC-d following treatment with TNP-470 and HPMA copolymer-TNP-470. (FIG. 18G) TNP-470 inhibits VPF/VEGF-induced MAPK phosphorylation in HMVEC-d. Densitometrical analysis is presented as percentage of band intensity compared to VEGF-stimulated control.

FIGS. 19A-19F show the effect of VPF/VEGF and RhoA signaling on HMVEC migration in vitro and on vessel permeability in vivo. (FIG. 19A) Migration assay was carried out in HMVEC (with 5 ng/ml VPF/VEGF stimulation) or HMVEC treated with TNP-470 and P-TNP-470 (1 ng/ml TNP-470-equivalent concentration). TNP-470 and P-TNP-470 inhibit basal and VPF/VEGF-induced migration of HMVEC-d. (FIG. 19C-FIG. 19D) TNP-470 and P-TNP-470 inhibit RhoA activation in HMVEC-d induced by VEGF (FIG. 19B), PAF (FIG. 19C) and histamine (FIG. 19D). Densitometrical analysis is presented as percentage of band intensity compared to VEGF-stimulated control. (FIG. 19E) TNP-470 and Y27632 inhibited both VEGF and CNF-1-induced vessel leakage. (FIG. 19F) Quantification of dye content in skin areas of the extravasation of Evan's blue dye at injection sites shown in (FIG. 19E). TNP-470 and Y27632 reduced both VEGF and CNF-1-induced vessel permeability to Evan's blue-albumin complex.

FIG. 20 shows a chematic model for proposed mechanism of TNP-470 inhibition of vessel permeability. TNP-470 inhibits migration and proliferation of endothelial cells and prevents VEGF-, PAF- and histamine-induced permeability. VEGF, PAF and histamine enhance vascular leakage by opening of inter-endothelial junctions, endothelial fenestration, generation of trans-endothelial gaps and transcytotic vesicles including VVO. Pretreatment with TNP-470 decreases the leakage via transcytotic vesicles. TNP-470 inhibited VPF/VEGF receptor-2 phosphorylation, [Ca²⁺]i and Rho A activation in vascular endothelium. This model suggests that TNP-470 transforms angiogenic and hyperpermeable vessels to a less leaky morphologic phenotype.

### DETAILED DESCRIPTION

We demonstrated in a mouse model that treatment with endostatin resulted in a significantly lower capillary leakage following intradermal injection of permeability-inducing agents (e.g., VEGF and platelet-activating factor (PAF)) compared with saline treated mice. These results suggest that the anti-tumor activity of endostatin might be explained in part by its anti-blood vessel permeability activity. Blood vessel permeability is associated with other diseases besides cancer such as vascular complications of diabetes such as diabetic retinopathy and nephropathy, nephrotic syndrome, vascular hypertension, burn edema, tumor edema, brain tumor edema, IL-2 therapy-associated edema, and other edema-associated diseases, for example, “Reperfusion” syndromes following ischemic injury in brain and heart, transplantation of organs, and surgery for removal of large tumors in the pelvis where major vessels must be occluded temporarily; Cerebral edema associated with brain tumors, head injury or stroke; Lymphedema associated with axillary lymph node dissection following mastectomy; and Allergic reactions associated with edema.

Thus, molecules that display anti-angiogenic activity, such as endostatin, can be used to prevent and treat pathologic blood vessel hyperpermeability in addition to their use in anti-cancer therapy. Such molecules may also be used to prevent the loss of endogenous angiogenic inhibitors or chemotherapeutic agents into the urine and thus are useful in the treatment of diseases or disorders involving abnormal angiogenesis including cancer.

In one aspect of the present invention there is provided a method of decreasing or inhibiting vascular hyperpermeability in an individual in need of such treatment. The method involves administering to the individual an effective amount of an antiangiogenic compound selected from the group consisting of endostatin, thrombospondin, angiostatin, tumstatin, arrestin, recombinant EPO, and polymer conjugated TNP-470. Preferably, the polymer is a HPMA copolymer.

Other angiogenesis inhibitors useful in the present invention include Taxane and derivatives thereof; interferon alpha, beta and gamma; IL-12; matrix metalloproteinases (MMP) inhibitors (e.g.,: COL3, Marimastat, Batimastat); EMD121974 (Cilengitide); Vitaxin; Squalamin; Cox2 inhibitors; PDGFR inhibitors (e.g., Gleevec); EGFR1 inhibitors (e.g., ZD1839 (Iressa), DSI774, SI1033, PKI166, IMC225 and the like); NM3; 2-ME2; Bisphosphonate (e.g., Zoledronate).

Taxane (paclitaxel) derivatives are disclosed in WO01017508, the disclosure of which is incorporated herein by reference.

Examples of inhibitors of matrix metalloproteinases include, but are not limited to, tetracycline derivatives and other non-peptidic inhibitors such as AG3340 (from Agouron), BAY 12-9566 (from Bayer), BMS-275291 (from Bristol-Myers Squibb) and CGS 27023A (from Novartis) or the peptidomimetics marimastat and Batimastat (from British Biotech), and the MMP-3 (stromelysin-1) inhibitor, Ac-RCGVPD-NH2 available from Calbiochem (San Diego, Calif.). See Hidalgo et al. 2001. J. Natl. Can. Inst. 93: 178-93 for a review of MMP inhibitors in cancer therapy.

As used herein the term “COX-2 inhibitor” refers to a non-steroidal drug that relatively inhibits the enzyme COX-2 in preference to COX-1. Preferred examples of COX-2 inhibitors include, but are no limited to, celecoxib, parecoxib, rofecoxib, valdecoxib, meloxicam, and etoricoxib.

In accordance with the present invention, fumagilin analogs other than TNP-470 may also be used. Such analogs include those disclosed in U.S. Pat. Nos. 5,180,738 and 4,954,496.

The antiangiogenic agent may be linked to a water soluble polymer having a molecular weight in the range of 100Da to 800 kD. The components of the polymeric backbone may comprise acrylic polymers, alkene polymers, urethanepolymers, amide polymers, polyimines, polysaccharides and ester polymers. Preferably the polymer is synthetic rather than being a natural polymer or derivative thereof. Preferably the backbone components comprise derivatised polyethyleneglycol and poly(hydroxyalkyl(alk)acrylamide), most preferably amine derivatised polyethyleneglycol or hydroxypropyl(meth)acrylamide-methacrylic acid copolymer or derivative thereof. A preferred molecular weight range is 15 to 40 kD.

The antiangiogenic agent and the polymer are conjugated by use of a linker, preferably a cleavable peptide linkage. Most preferably, the peptide linkage is capable of being cleaved by preselected cellular enzymes. Alternatively, an acid hydrolysable linker could comprise an ester or amide linkage and be for instance, a cis-aconityl linkage. A pH sensitive linker may also be used.

Cleavage of the linker of the conjugate results in release of an active antiangiogenic agent. Thus the antiangiogenic agent must be conjugated with the polymer in a way that does not alter the activity of the agent. The linker preferably comprises at least one cleavable peptide bond. Preferably the linker is an enzyme cleavable oligopeptide group preferably comprising sufficient amino acid units to allow specific binding and cleavage by a selected cellular enzyme. Preferably the linker is at least two amino acids long, more preferably at least three amino acids long.

Preferred polymers for use with the present invention are HPMA copolymers with methacrylic acid with pendent oligopepticle groups joined via peptide bonds to the methacrylic acid with activated carboxylic terminal groups such as paranitrophenyl derivatives.

In a preferred embodiment the polymeric backbone comprises a hydroxyalkyl(alk)acrylamide methacrylamide copolymer, most preferably a copolymer of N-(2-hydroxypropyl)methacrylamide (HPMA) copolymer. Such polymers and methods of conjugation are disclosed in WO 01/36002.

In addition, antiangiogenic agent polymer conjugates of use in the present invention are disclosed in WO 03/086382.

A disease associated with vascular permeability for treatment with the present invention includes vascular complications of diabetes such as non-proliferative diabetic retinopathy and nephropathy, nephrotic syndrome, pulmonary hypertension, burn edema, tumor edema, brain tumor edema, IL-2 therapy-associated edema, and other edema-associated diseases.

Tight junctions regulate endothelial cell permeability and create an intramembrane diffusion fence. Tight junctions form discrete sites of fusion between the outer plasma membrane of adjacent cells. The tight junctions are complexes of molecules that build, associated with, or regulate the tight junction function. The junctions are composed of three regions: the integral membrane proteins, including, but not limited to, occludin and claudin; the cytoplasmic proteins, including, but not limited to, zonula occludens (ZO)-1, -2, -3; and proteins associated with tight junctions, including, but not limited to, catenins, cingulin and p130. Recent studies have shown that VEGF interferes with tight junction assembly via induction of rapid phosphorylation of tight junction proteins occludin and ZO-1, resulting in dislocation of these proteins from the cell membrane. VEGF was also shown to decrease the expression of occludin. We show in the examples below that interference with or destabilization of tight junction proteins increases vascular permeability and ultimately causes hyperpermeability. Therefore, stabilization of the tight junction proteins using compounds which inhibit endothelial cell proliferation and migration in vitro or otherwise repress tumor growth would be useful in the treatment or prevention of diseases associated with vascular hyperpermeability.

Compounds such as endostatin, thrombospondin, angiostatin, tumstatin, arrestin, recombinant EPO, and TNP-470 are widely available commercially. Those compounds that are not commercially available can be readily prepared using organic synthesis methods known in the art.

Whether or not a particular compound, in accordance with the present invention, can treat or prevent diseases associated with hyperpermeability can be determined by its effect in the mouse model as shown in the Examples below. Compounds capable of preventing or treating non-proliferative diabetic retinopathy can be tested by in vitro studies of endothelial cell proliferation and in other models of diabetic retinopathy, such as Streptozotocin. In addition, color Doppler imaging can be used to evaluate the action of a drug in ocular pathology (Valli et al., Ophthalmologica 209 (13): 115-121 (1995)). Color Doppler imaging is a recent advance in ultrasonography, allowing simultaneous two-dimension imaging of structures and the evaluation of blood flow. Accordingly, retinopathy can be analyzed using such technology.

The compounds useful in the prevention and treatment methods of the present invention can be administered in accordance with the present inventive method by any suitable route. Suitable routes of administration include systemic, such as orally or by injection or topical. The manner in which the therapeutic compound is administered is dependent, in part, upon whether the treatment of a disease associated with vascular hyperpermeability, including non-proliferative retinopathy is prophylactic or therapeutic. For example, the manner in which the therapeutic compound is administered for treatment of retinopathy is dependent, in part, upon the cause of the retinopathy. Specifically, given that diabetes is the leading cause of retinopathy, the effective compound can be administered preventatively as soon as the pre-diabetic retinopathy state is detected.

Thus, to prevent non-proliferative retinopathy that can result from diabetes, the effective compound is preferably administered systemically, e.g., orally or by injection. To treat non-proliferative diabetic retinopathy, the effective compound can be administered systemically, e.g., orally or by injection, or intraocularly. Other routes such as periocular (e.g., subTenon's), subconjunctival, subretinal, suprachoroidal and retrobulbar can also be used in the methods of the present invention. The effective compound is preferably administered as soon as possible after it has been determined that an individual is at risk for retinopathy (preventative treatment) or has begun to develop retinopathy (therapeutic treatment). Treatment will depend, in part, upon the particular effective compound used, the amount of the effective compound administered, the route of administration, and the cause and extent, if any, of retinopathy realized.

One skilled in the art will appreciate that suitable methods of administering an effective compound, which is useful in the present inventive method, are available. Although more than one route can be used to administer the effective compound, a particular route can provide a more immediate and more effective reaction than another route. Accordingly, the described routes of administration are merely exemplary and are in no way limiting.

The dose of the effective compound administered to an individual, particularly a human, in accordance with the present invention should be sufficient to effect the desired response in the animal over a reasonable time frame. One skilled in the art will recognize that dosage will depend upon a variety of factors, including the strength of the particular compound employed, the age, condition or disease state (e.g., the amount of the retina about to be affected or actually affected by retinopathy), and body weight of the individual. The size of the dose also will be determined by the route, timing and frequency of administration as well as the existence, nature, and extent of any adverse side effects that might accompany the administration of a particular compound and the desired physiological effect. It will be appreciated by one of ordinary skill in the art that various conditions or disease states, in particular, chronic conditions or disease states, may require prolonged treatment involving multiple administrations.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. The present inventive method will typically involve the administration of from about 1 mg/kg/day to about 500 mg/kg/day, preferably from about 10 mg/kg/day to about 200 mg/kg/day, if administered systemically. Intraocular administration typically will involve the administration of from about 0.1 mg total to about 5 mg total, preferably from about 0.5 mg total to about 1 mg total.

Compositions for use in the present inventive method preferably comprise a pharmaceutically acceptable carrier and an amount of a compound sufficient to treat or prevent diseases associated with vascular hyperpermeability and non-proliferative retinopathy. The carrier can be any of those conventionally used and is limited only by chemico-physical considerations, such as solubility and lack of reactivity with the compound, and by the route of administration. It will be appreciated by one of ordinary skill in the art that, in addition to the following described pharmaceutical compositions, the compound used in the methods of the present invention can be formulated as polymeric compositions, inclusion complexes, such as cyclodextrin inclusion complexes, liposomes, microspheres, microcapsules and the like (see, e.g., U.S. Pat. Nos. 4,997,652, 5,185,152 and 5,718,922).

The effective compound used in the present invention can be formulated as a pharmaceutically acceptable acid addition salt. Examples of pharmaceutically acceptable acid addition salts for use in the pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, and arylsulphonic, for example p-toluenesulphonic, acids.

The pharmaceutically acceptable excipients described herein, for example, vehicles, adjuvants, carriers or diluents, are well-known to those who are skilled in the art and are readily available to the public. It is preferred that the pharmaceutically acceptable carrier be one which is chemically inert to the compound used and one which has no detrimental side effects or toxicity under the conditions of use.

The choice of excipient will be determined in part by the particular compound, as well as by the particular method used to administer the composition. Accordingly, there is a wide variety of suitable formulations of the pharmaceutical composition of the present invention. The following formulations are merely exemplary and are in no way limiting.

Injectable formulations are among those that are preferred in accordance with the present inventive method. The requirements for pharmaceutically effective carriers for injectable compositions are well-known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986)). It is preferred that such injectable compositions be administered intramuscularly, intravenously, or intraperitoneally.

Topical formulations are well-known to those of skill in the art. Such formulations are suitable in the context of the present invention for application to the skin. The use of patches, corneal shields (see, e.g., U.S. Pat. No. 5,185,152), and ophthalmic solutions (see, e.g., U.S. Pat. No. 5,710,182) and ointments, e.g., eye drops, is also within the skill in the art.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The effective compound for use in the methods of the present invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride, with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Oils, which can be used in parenteral formulations include petroleum, animal, vegetable, or synthetic oils. Specific examples of oils include peanut, soybean, sesame, cottonseed, corn, olive, petrolatum, and mineral.

Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metals, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-p-aminopropionates, and 2-alkyl-imidazoline quaternary ammonium salts, and (e) mixtures thereof.

The parenteral formulations will typically contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17.

The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Such compositions can be formulated as intraocular formulations, sustained-release formulations or devices (see, e.g., U.S. Pat. No. 5,378,475). For example, gelantin, chondroitin sulfate, a polyphosphoester, such as bis-2-hydroxyethyl-terephthalate (BHET), or a polylactic-glycolic acid (in various proportions) can be used to formulate sustained-release formulations. Implants (see, e.g., U.S. Pat. Nos. 5,443,505, 4,853,224 and 4,997,652), devices (see, e.g., U.S. Pat. Nos. 5,554,187, 4,863,457, 5,098,443 and 5,725,493), such as an implantable device, e.g., a mechanical reservoir, an intraocular device or an extraocular device with an intraocular conduit (e.g., 100 mu-1 mm in diameter), or an implant or a device comprised of a polymeric composition as described above, can be used.

The present inventive method also can involve the co-administration of other pharmaceutically active compounds. By “co-administration” is meant administration before, concurrently with, e.g., in combination with the effective compound in the same formulation or in separate formulations, or after administration of the effective compound as described above. For example, corticosteroids, e.g., prednisone, methylprednisolone, dexamethasone, or triamcinalone acetinide, or noncorticosteroid anti-inflammatory compounds, such as ibuprofen or flubiproben, can be co-administered. Similarly, vitamins and minerals, e.g., zinc, anti-oxidants, e.g., carotenoids (such as a xanthophyll carotenoid like zeaxanthin or lutein), and micronutrients can be co-administered. Other various compounds that can be co-administered include sulphonylurea oral hypoglycemic agent, e.g., gliclazide (non-insulin-dependent diabetes), halomethyl ketones, anti-lipidemic agents, e.g., etofibrate, chlorpromazine and spinghosines, aldose reductase inhibitors, such as tolrestat, sorbinil or oxygen, and retinoic acid and analogues thereof (Burke et al., Drugs of the Future 17(2): 119-131 (1992); and Tomlinson et al., Pharmac. Ther. 54: 151-194 (1992)). Those patients that exhibit systemic fluid retention, such as that due to cardiovascular or renal disease and severe systemic hypertension, can be additionally treated with diuresis, dialysis, cardiac drugs and antihypertensive agents.

In yet another aspect of the invention there is provided a method of screening for compounds that stabilize tight junction proteins. The method involves culturing endothelial cells in the presence of a test compound, contacting the cultured endothelial cells with a tight junction protein, and assessing whether the test compound stabilized the tight junction protein. The compound that stabilizes the tight junction protein is indicative of an anti-permeability and/or an anti-angiogenic compound. The tight junction protein contemplated by the present invention includes integral membrane proteins, cytoplasmic proteins, and proteins associated with tight junctions. More particularly, the tight junction proteins include occludin, claudin, zonula occludens (ZO)-1, -2, -3, catenins, cingulin and p130. One embodiment of the method of screening for compounds that stabilize tight junction proteins is described in the Examples section below.

In a further aspect of the invention there is provided a method of screening for compounds that affect vascular permeability. The method, one embodiment of which is described below in the Examples section of the application, involves assaying endothelial cells on a permeable substrate (e.g., a collagen coated inserts of “Transwells”), contacting the assay with a test compound (e.g., an antiangiogenic compound such as endostatin), treating the assay with a marker (e.g., FITC label) and a permeability-inducing agent (e.g., vascular endothelial growth factor (VEGF) and platelet-activating factor (PAF) among others), and measuring the rate of diffusion of the marker compare to control. Compounds that are found to affect vascular permeability can be further tested for anti-tumor activity using existing methods.

In another aspect of the present invention there is provided a method for assessing bioeffectiveness of an antiangiogenic compound in a patient being treated with such compound. The method involves administering to the patient an intradermal injection of histamine before treating the patient with the antiangiogenic compound and measuring a histamine-induced local edema. Then, treating the patient with the antiangiogenic compound, and again administering to said patient an intradermal injection of histamine subsequent to treating the patient with the antiangiogenic compound and measuring the histamine-induced local edema. A decrease in the measurement of the histamine-induced local edema compared to that seen before the treatment with the antiangiogenic compound indicates that the compound is bioeffective.

The present invention also provides an alternative method for assessing a bioeffectiveness of an antiangiogenic compound in a patient being treated with such compound. It has been observed that patients suffering from diseases associated with vascular hyperpermeability have higher protein levels in the urine compare to a control group. The method involves measuring a level of a protein in a bodily fluid of the patient (e.g., blood or urine) before treating the patient with the antiangiogenic compound, then, treating the patient with the antiangiogenic compound and measuring the level of the protein in the bodily fluid of the patient. A decrease in the level of the protein in the bodily fluid compare to the pre-treatment level indicates that the compound inhibits vascular permeability and is bioeffective.

Finally, the present invention provides an article of manufacture which includes packaging material and a pharmaceutical agent contained within the packaging material. The packaging material includes a label which indicates said pharmaceutical may be administered, for a sufficient term at an effective dose, for treating and/or preventing a disease associated with vascular permeability. The pharmaceutical agent is selected from the group consisting of endostatin, thrombospondin, angiostatin, tumstatin, arrestin, recombinant EPO and polymer conjugated TNP-470. The disease associated with vascular permeability includes, but not limited to, non-proliferative diabetic retinopathy, diabetic nephropathy, nephrotic syndrome, pulmonary hypertension, burn edema, tumor edema, brain tumor edema, IL-2 therapy-associated edema, and other edema-associated diseases.

The invention will be further characterized by the following examples which are intended to be exemplary of the invention.

### EXAMPLES

### Example 1

Effect of Endostatin on Vascular Permeability and Hyperpermeability:

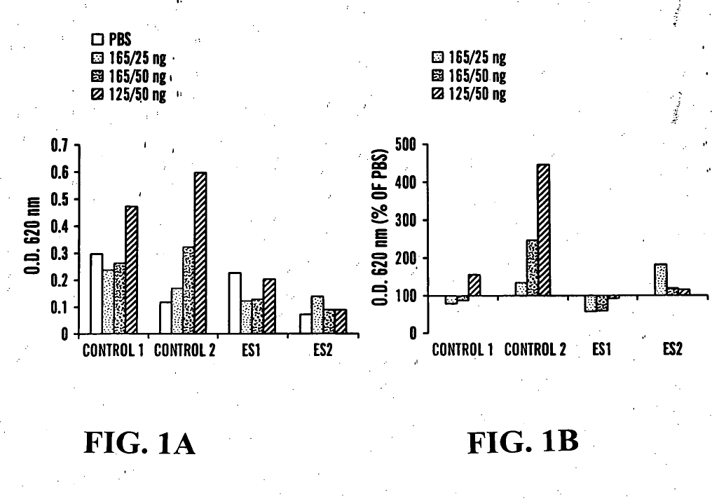

The antiangiogenic factor (endostatin) was injected intraperitoneally to FVB/NJ mice for 4 days. Immediately after the last injection, mice were anasthesized and received intravenous injection of 100 μl Evans Blue dye (1% in PBS). Subsequently, different amounts of VEGF₁₆₅, VEGF₁₂₁ or saline were injected intradermaly. After 20 minutes, mice were sacrificed and skin flap from the back was removed and photographed. Skin samples from the injection sites were excised and incubated in formamide for 5 days in order to extract the dye and O.D. was measured at 620 nm. Macroscopic examination of skin flaps from control mice showed massive extravasation of Evans Blue dye at the VEGF injection sites. VEGF₁₂, had a stronger hyperpermeability activity that VEGF₁₆₅ and there was not much difference between 25 and 50 ng/ml VEGF₁₆₅. Mice treated with the antiangiogenic factor had an overall lower dye leakage than the control and had minor induction of hyperpermeability by VEGF injection. Quantitative analysis of Evans Blue dye extravasation (FIG. 1) confirmed the lower skin capillary permeability of the antiangiogenic factor-treated mice and indicated the weak permeability-inducing effect of VEGF in these mice. These results suggest that the antiangiogenic factor may have a general anti-vascular permeability effects as well as inhibition of VEGF-induced hyperpermeability.

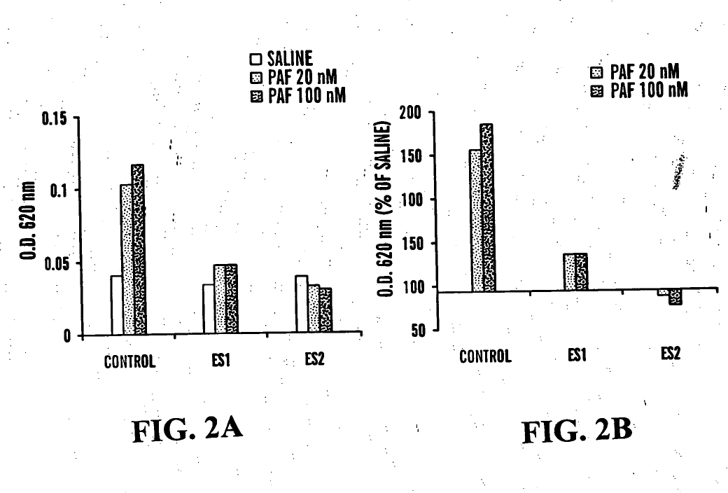

In order to test if the effects of the antiangiogenic factor (endostatin) on vascular permeability is VEGF-specific, we have tested the effects of intradermal injection of platelet-activating factor (PAF) in Nude mice that were previously injected with the antiangiogenic factor and in control mice, as described above. Macroscopic examination of skin flaps confirmed that the antiangiogenic factor inhibits vascular permeability. The antiangiogenic factor also repressed PAF-induced vascular permeability. Quantitative analysis of Evans Blue dye extravasation (FIG. 2) confirmed the lower skin capillary permeability of the antiangiogenic factor-treated mice compared with control and the lack of PAF-induced hyperpermeability in these mice. Thus, it seems that the anti-vascular hyperpermeability effect of the antiangiogenic factor is not restricted to VEGF-induced permeability and affects other mediators of blood vessel permeability such as PAF.

Duration of Exposure to Antiangiogenic Factors to Inhibit Blood Vessel Permeability:

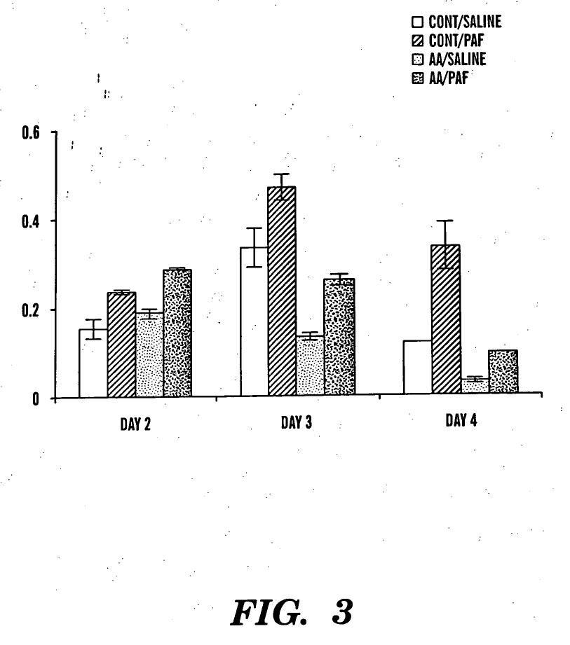

In order to test if continuous exposure to the antiangiogenic factor (endostatin) is required to repress blood vessel permeability, mice (SCID) were anesthetized and “Alzet” pumps loaded with the antiangiogenic factor or saline were implanted intraperitoneally. The pumps release 1 μl the antiangiogenic factor per hour. Skin vessel permeability using Evans Blue dye was performed as described above. Saline and the antiangiogenic factor treated mice were examined 2, 3 and 4 days after pump implantation, as described above (FIG. 3). At day two there was no significant difference between blood vessel permeability in response to PAF injection between saline and the antiangiogenic factor treated mice. In both groups, PAF injection induced higher vessel permeability than saline injection. In contrast, at days three and four both saline and PAF injections in saline treated mice induced significantly higher vessel permeability than in the antiangiogenic factor treated mice. However, in both groups PAF injection induced higher vessel permeability than saline injection. These results indicate that at least 3 days treatment with the antiangiogenic factor were required to reduce skin vessel permeability. Taken together, the results suggest that continuous exposure of the vasculature to the antiangiogenic factor may prevent blood vessel hyperpermeability and leakage of plasma proteins to surrounding tissue. Since the tumor vessels are continuously permeabilized and plasma proteins contained within the tumor support its vascularization the anti-permeability effect of the antiangiogenic factor offers a possible mechanism for its anti-tumor activity.

Endostatin Inhibits Diffusion Through Endothelial Cell Monolayer in Vitro:

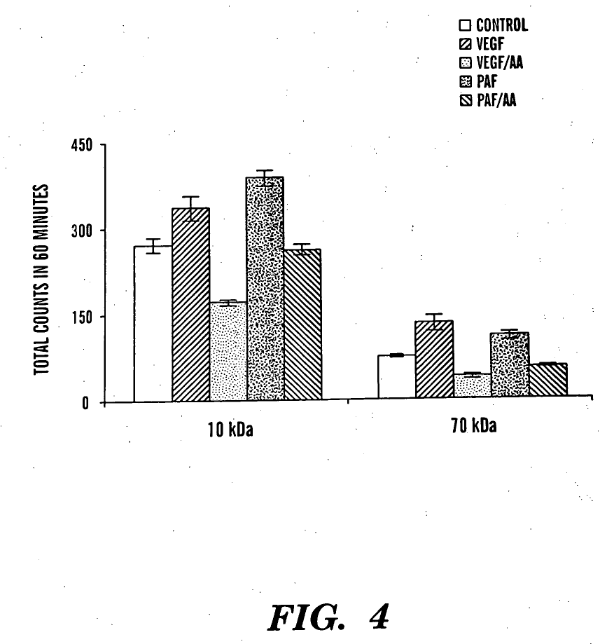

The effects of the antiangiogenic factor (endostatin) on skin vessel permeability in vivo were tested in an in vitro diffusion model designed to mimic blood vessel permeability. Bovine capillary endothelial cells (BCE) were seeded in collagen coated inserts of “Transwells” and grown to confluence. The antiangiogenic factor was added every 24 hours. Four days later the inserts were washed with BCE culture medium and the following tracers and permeability regulators were added to the inserts. Half of the inserts received 5 mg/ml FITC-labeled dextran 10 kDa and the other half received 5 mg/ml FITC-labeled dextran 70 kDa. In addition, some inserts received 50 ng/ml VEGF₁₆₅ or 100 nM PAF. Control inserts received BCE culture medium with fluorescent tracers only. The fluorescence in the lower wells was measured after 10, 20, 30, 45 and 60 minutes by transferring the inserts into new wells. The sum of fluorescent count over 60 minutes showed higher values in cells treated with VEGF₁₆₅ and PAF compared with control cells (FIG. 4). The number of counts in VEGF₁₆₅ and PAF treated cells was observed with 10 kDa and 70 kDa dextrans. Cells that were pre-treated with the antiangiogenic factor showed significantly lower fluorescent counts then control, VEGF₁₆₅-treated and PAF-treated cells in both dextran sizes. The reduction in fluorescent counts in the antiangiogenic factor pre-treated cells was more pronounced in the diffusion of 70 kDa dextran compared with that of 10 kDa dextran. These results indicate that the in vitro diffusion system responds positively to permeability inducing factors such as VEGF and PAF.

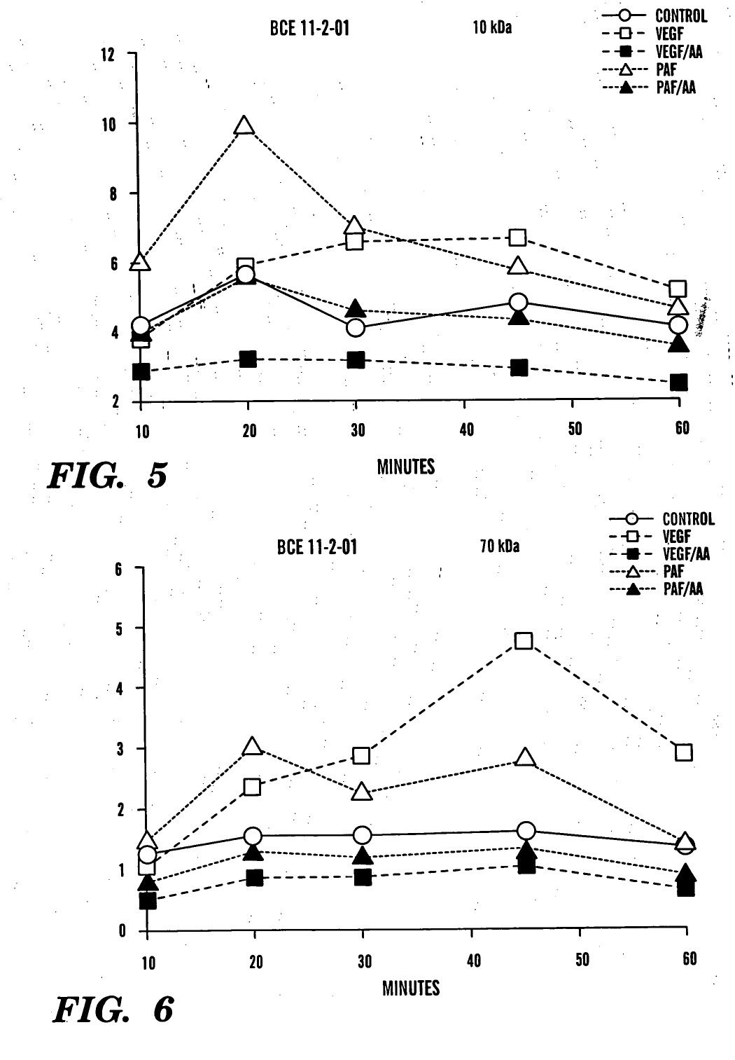

Moreover, the results indicate that the antiangiogenic factor treatment significantly reduces the diffusion of large molecules through EC monolayer. In order to follow the kinetic of the diffusion process, the flow of the tracer was calculated as fluorescent counts per minute (FIGS. 5 and 6). Using 10 kDa dextran (FIG. 5), PAF progressively increased the flow up to 20 minutes and then the flow was reduced and reached similar levels as in the control cells. VEGF₁₆₅ had a similar effect but it reached the maximum flow at 45 minutes and the flow was lower than in PAF-treated cells. In contrast, the flow in control cells was constant and was lower than that observed in PAF and VEGF₁₆₅-treated cells. The results obtained with 70 kDa dextran (FIG. 6) were similar to those of the 10 kDa dextran, only that when using 70 kDa dextran VEGF₁₆₅-treatment resulted in higher flow than in PAF treatment. The antiangiogenic factor pre-treatment resulted in significant reduced flow of the 10 kDa and the 70 kDa dextrans.

Like control cells, the antiangiogenic factor-treated cells had a constant flow during the 60 minutes period. The flow in the antiangiogenic factor-treated cells was lower than that of control cells. Taken together, these results indicate that the antiangiogenic factor treatment results in slower diffusion through EC monolayer. These results suggest that the effect of the antiangiogenic factor on diffusion of large molecules may explain it inhibition of blood vessel permeability. In addition, the in vitro diffusion system can be used to test the effect of anti-angiogenesis and other molecules on blood vessel permeability.

Endostatin Inhibits Swelling of the Lung Tissue

Dilation of the lung tissue may result in lung dysfunction and development of pulmonary hypertension. Mice injected with micro-encapsulated cells producing VEGF (approximately 0.5×10⁶ cells per mouse) developed thickened lung parenchyma 5 days after injection. At a higher magnification we observed generation of several cell layers between the alveoli compared with one layer of cells in mice injected with micro-encapsulated control cells or with micro-encapsulated cells producing endostatin (Endost). In addition, we observed accumulation of extracellular matrix (usually stained pink with H & E staining) in the lung tissue of VEGF-treated mice, suggesting that high levels of circulating VEGF might induce leakage of plasma proteins into the lung tissue. In contrast, the lungs of mice received VEGF producing cells together with endostatin producing cells (0.5×10⁶ encapsulated cells of each) appeared similar to the lungs of mice injected with control cells and had fewer cell layers and no accumulation of extracellular matrix. These results indicate that endostatin may prevent leakage of plasma proteins into the lung tissue and the accumulation of extracellular matrix in the tissue. Moreover, treatment with endostatin reduced the number of cell layers between the alveoli and the lungs of mice that were treated with endostatin appeared similar to control mice. Therefore, endostatin appears to block the swelling of lung tissue and may be used for treatment of pulmonary hypertension.

Endostatin Increases the Assembly of Tight Junction Proteins:

Bovine capillary endothelial cells (BCE) were cultured in the presence of 0.2, 0.5 and 2 μg/ml human endostatin for three days. The cells were fixed and immunostained with anti-β-catenin, occludin, and ZO-1 antibodies (Zymed Laboratories, CA). The staining was developed using FITC-conjugated secondary antibodies and visualized under fluorescent microscopy. Immunostaining for β-catenin marked the cell borders and was more intense when two cells contacted each other. The cell boundary β-catenin staining was intensified in the presence of 0.2 μg/ml endostatin and further intensified in the presence of 0.5 μg/ml endostatin. There was no difference in β-catenin staining between 0.5 and 2.0 μg/ml endostatin. Immunostaining for occludin, in the absence of endostatin, did not show any cell borders demarcation, rather the cell nuclei were stained. However, in the presence of 0.5 and 2.0 μg/ml endostatin cell boundaries were observed mostly when two cells contacted each other. Similar results were obtained with ZO-1 immunostaining. Cells boundaries were only visible in the presence of 0.2-2.0 μg/ml endostatin. These results indicate that immunostaining for tight junction proteins in enhanced in the presence of endostatin and suggest that endostatin may support assembly and stabilization of tight junctions. This is the first documentation of the effects of endostatin on tight junctions that may explain, in part, the mechanism of its antiangiogenic activities. Similar experiments were performed in which BCE were incubated in the presence and absence of 0.5 μg/ml endostatin for 3 days followed by stimulation with PAF for 20 minutes. The cells were fixed and immunostained with anti-α-catenin, occludin, and ZO-1 antibodies (Zymed Laboratories, CA), as described above. PAF treatment significantly reduced the staining intensity of anti-α-catenin, occludin, and ZO-1 only in control cells but not in endostatin-treated cells. These results point to tight junction proteins as possible target for anti-permeability and anti-cancer therapeutic approaches.

The Use of Histamine-Induced Wheal and Flare Assays to Test the Activity of Antiangiogenic Treatment:

Antiangiogenic treatment has entered into clinical trials recently. Molecules that are tested in phase 1 and 2 clinical trials include endostatin, angiostatin, TNP-470, thalidomide, anti-VEGF antibodies, PTK787, SU-5416, SU-6668 and others. Our results indicating that endostatin treatment reduces skin blood vessel permeability support that this test can be used to determine the efficiency of endostatin (and other antiangiogenic agent) treatment in human patients. Mice that received endostatin for several days had lower diffusion of Evans blue from the skin capillaries in response to intradermal VEGF and PAF injection compared with normal mice. The existing test of histamine-induced wheal and flare in skin can be used in order to test bioactivity of endostatin and other antiangiogenic factors. Intradermal injection of histamine leads to the formation of local adema (flare) due to blood vessel hyperpermiability. Humans receiving endostatin and other antiangiogenic factors will have a reduced zone of edema due to the anti-permeability activity. This test will serve as an early surrogate marker for the bioactivity of endostatin and other antiangiogenic factors and help to determine the treatment's efficiency in individual patients.

### Example 2

### HPMA copolymer-TNP-470 Inhibts the Proliferation of BCE Cells and Chick Aortic Rings In Vitro

Synthesis of HPMA Copolymer-TNP-470 Conjugate:

TNP-470 was conjugated to HPMA copolymer-Gly-Phe-Leu-Gly-ethylendiamine via nucleophilic attack on the α-carbonyl on the TNP-470 releasing the chlorine. Briefly, HPMA copolymer-Gly-Phe-Leu-Gly-ethylendiamine (100 mg) was dissolved in DMF (1.0 ml). Then, TNP-470 (100 mg) was dissolved in 1.0 ml DMF and added to the solution. The mixture was stirred in the dark at 4° C. for 12 h. DMF was evaporated and the product, HPMA copolymer-TNP-470 conjugate was redissolved in water, dialyzed (10 kDa MWCO) against water to exclude free TNP-470 and other low molecular weight contaminants, lyophilized and stored at −20° C. Reverse phase HPLC analysis using a C18 column, was used to characterize the conjugate.

BCE Proliferation Assay:

Bovine adrenal capillary endothelial cells were seeded on gelatinized plates (15,000/well). Following 24 h incubation, cells were challenged with free and conjugated TNP-470, and bFGF (1 ng/ml) was added to the medium. Cells were counted after 72 h.

Chick Aortic Ring Assay:

Aortic arches were dissected from day-14 chick embryos and cut into cross-sectional fragments, everted to expose the endothelium, and explanted in Matrigel. When cultured in serum-free MCDB-131 medium, endothelial cells outgrow and three-dimensional vascular channel formation occurred within 2-48 hours. Free and conjugated TNP-470 were added to the culture.

Miles Assay:

One of the problems with angiogenesis-dependent diseases is increased vessel permeability (due to high levels of VPF) which results in edema and loss of proteins. A decrease in vessel permeability is beneficial in those diseases. We have found, using the Miles assay (Claffey, et al., Cancer Res, 56:172-181 (1996)), that free and bound TNP-470 block permeability. Briefly, a dye, Evans Blue (1% in PBS), was injected i.v. to anesthesized mice. After 10 min, human recombinant VEGF₁₆₅ (50 ng/50 μl) was injected intradermally into the back skin. Leakage of protein-bound dye was detected as blue spots on the underside of the back skin surrounding the injection site. After 20 min mice were euthanized. Then, the skin was excised, left in formamide for 5 days to be extracted and the solution read at 620 nm. Putative angiogenesis inhibitors such as free and conjugated TNP-470 were injected daily 3 days (30 mg/kg/day) prior to the VEGF challenge. The same was repeated on tumor-bearing mice to evaluate the effect of angiogenesis inhibitors on tumor vessel permeability.

Hepatectomy:

C57 black male mice underwent a ⅔ hepatectomy through a midline incision after general anesthesia with isoflourane. Free and conjugated TNP-470 (30 mg/kg) was given s.c. every other day for 8 days beginning on the day of surgery. The liver was harvested on the 8^{th} day, weighed and analyzed for histology.

Results:

HPMA copolymer-TNP-470 conjugate was synthesized, purified and characterized by HPLC. Free TNP-470 had a peak at a retention time of 13.0 min while the conjugate had a wider peak at 10.0 min. No free drug was detected following purification.

TNP-470 is not water-soluble but became soluble following conjugation with HPMA copolymer. To evaluate the biological activity of HPMA-TNP-470, the following assays were performed:

BCE proliferation: BCE cell growth was inhibited by TNP-470 and BPMA copolymer-TNP-470 similarly when challenged with bFGF (data not shown).

Aortic ring assay: Free and conjugated TNP-470 reduced the number and length of vascular sprouts and showed efficacy at 50 pg/ml and completely prevented outgrowth at 100 pg/ml. Untreated aortic ring shows abundant sprouting.

Hepatectomy: Following ⅔ hepatectomy, control mice regenerated their resected liver mass to their pre-operative levels (˜1.2 g) by post-operative day 8. Mice treated with free TNP-470 or different doses of its polymer-conjugated form inhibited the regeneration of the liver and retained it at an average size of 0.7 g on post-operative day 8. HPMA-TNP-470 conjugate had a similar effect even when given at a single does on the day of hepatectomy showing a longer circulation time and sustained release from the polymer at the site of proliferating endothelial cells. Because liver regeneration is regulated by endothelial cells growth, it is expected that the same effect will be on proliferating endothelial cells in tumor issue.

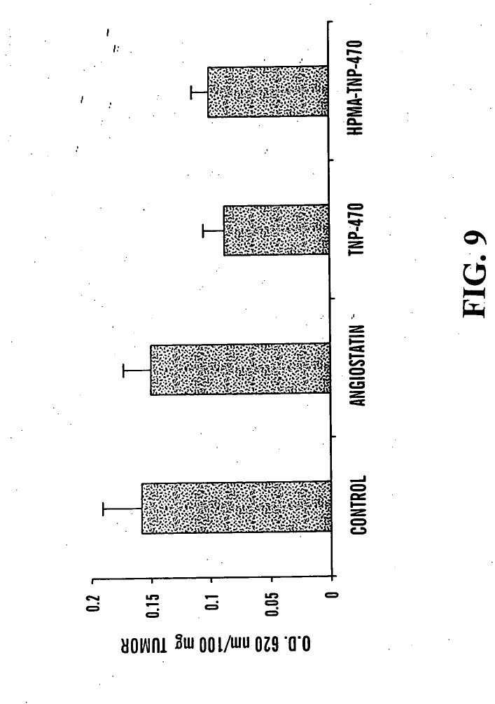

Miles assay: We have compared free and conjugated TNP-470 to other angiogenesis inhibitors in the Miles assay. We have found that free TNP-470 and HPMA copolymer-TNP-470 had similar inhibitory effect on VEGF induced vessel permeability as opposed to the control groups and indirect angiogenesis inhibitors such as Herceptin and Thalidomide. Free and conjugated TNP-470 at 30 mg/kg/day for three days also decreased tumor vessel permeability in A2058 human melanoma-bearing mice (FIG. 9).

Conclusions:

HPMA copolymer-TNP-470 inhibited the proliferation of BCE cells and chick aortic rings in vitro. In vivo the conjugate had a similar effect as the free TNP-470 on liver regeneration following hepatectomy. This suggests that it retained its inhibitory activity when released from the polymeric conjugate by lysosomal enzymatic cleavage of the tetrapeptide (Gly-Phe-Leu-Gly) linker in the proliferating endothelial cells.

### Example 3

Effects of TNP-470 on Vascular Permeability

Experimental Procedures

Materials

A random copolymer of HPMA and methacyrloyl-Gly-Phe-Leu-Gly-p-nitrophenyl ester (HPMA copolymer-MA-GFLG-ONp) incorporating approximately 10 mol % of the MA-GFLG-ONp monomer units was prepared as previously reported (Rihova et al., 1989) and this polymeric precursor was supplied in its ethylenediamine (en) derivative form by Polymer Laboratories (UK). The HPMA copolymer-GFLG-en had a Mw of 31,600 Da and polydispersity (PD) of 1.66. TNP-470 was kindly provided by Douglas Figg from the NCI (USA) and Takeda Chemical Industries Ltd. (Japan). HPMA copolymer-TNP-470 was synthesized as previously described (Satchi-Fainaro et al., 2004) and batches had ˜10% w/w of TNP-470 content. VEGF₁₆₅ was a gift from the NIH (Bethesda, Calif.). Bovine serum albumin (BSA), dimethylformamide (DMF), formamide, Evan's blue, histamine and oxazolone (4-Ethoxymethylene-2-phenyloxazolone) were from Sigma (St Louis, Mo.). Platelet activating factor (PAF) was from Biomol (Plymouth Meeting, Pa., USA), Vivacell 70 ml dialysis system (10 kDa MW cut-off PES) was from VivaScience (USA). Isoflurane was purchased from Baxter Healthcare Corporation (USA). Matrigel basement membrane matrix (from Engelbreth-Holm-Swarm mouse tumor) was purchased from Becton Dickinson (USA). Avertin was purchased from Fisher (USA). Thalidomide was from Celgene (USA). Human and mouse VPF/VEGF quantikine ELISA kits were purchased from R & D Systems Inc. (Minneapolis, Minn., USA). Angiostatin was from EntreMed (USA). Anti-Erb B-2 antibody (Herceptin) was from Genentech (USA). IL-2 was a gift from Dr. Steven A. Rosenberg (NIH). Inserts of Transwells were from Costar. Rabbit polyclonal antibody against RhoA, Anti-Flk-1mouse monoclonal IgG1 and Anti-phosphotyrosine (Ptyr) mouse monoclonal IgG2b were purchased from Santa Cruz Biotechnology (Santa Cruz, Calif.). Anti-Phospho-p44/42 MAPK(Thr202/Tyr204) mouse monoclonal antibody and Anti-p44/42 MAP Kinase rabbit polyclonal antibody were from Cell Signaling Technology, Inc. Y27632 was from Calbiochem (San Diego, Calif.). His-CNF1 plasmid was a gift from Melody Mills (Uniformed Services University of Health Sciences, Maryland, USA) and was expressed in E-Coli, and recombinant CNF-1 purified with the QIAGEN kit. Glutathione-S-transferase (GST)-Rhotekin Rho binding domain (TRBD) fusion protein was provided by Dr. Martin Schwartz (Scripps Institute) (Ren et al., 1999).

Cell Culture

A2058 human melanoma cells, U87 human glioblastoma, BXPC3, LLC, MCF-7, MDA-MB-231 cells were from the American Type Culture Collection, ATCC (Manassas, Va.). Cells were maintained in Dulbecco's modified Eagle's medium (DMEM) or RPMI medium 1640 (for BXPC3 cells) containing 10% inactivated fetal bovine serum (Life Technologies, Inc.), 0.29 mg/ml L-glutamine, 100 units/ml penicillin and 100 μg/ml streptomycin (Gibco) in a humidified 5% CO₂ incubator at 37° C. Human dermal microvascular endothelial cells (HMVEC-d) and human umbilical vascular endothelial cells (HUVEC) were obtained from Clonetics/BioWhittaker (Walkersville, Md.) and grown according to the manufacturer's protocol in EGM-2 MV medium or EGM, respectively. EGM-2 MV bullet Kit (contains FBS 5%, Hydrocortisone, human fibroblast growth factor-basic with heparin (hFGF-B), human recombinant insulin-like growth factor (R3-IGF), human recombinant epidermal growth factor (hEGF), VEGF, ascorbic acid, gentamycin, amphotericin-B) and endothelial cell basic medium (EBM-2) were purchased from Clonetics (San Diego, Calif.).

Mice

C57B1/6J mice were purchased from Jackson Laboratories (USA) and CB-17 SCID mice were purchased from Massachusetts General Hospital (USA). All animal procedures were performed in compliance with Boston Children's Hospital guidelines and approved protocols by the Institutional Animal Care and Use Committee.

Miles Vascular Permeability Assay

SCID mice were injected subcutaneously (s.c.) with TNP-470 or HPMA copolymer-TNP-470 (30 mg/Kg TNP-equiv.) for three days, with Y27632 (50 nM s.c.) for 11 days, with angiostatin (200 mg/kg/day s.c.) for five days, or with saline (250 μl s.c.) for 5 days (n=12) prior to performing the Miles assay (Claffey et al., 1996; Miles and Miles, 1952; Streit et al., 2000). Briefly, Evan's blue dye (100 μl of a 1% solution in 0.9% NaCl) was injected intravenously (i.v.) into mice. After 10 minutes, 50 μl of human VEGF₁₆₅ (1 ng/μl), PAF (100 μM), CNF-1 (100 ng), histamine (1.2 μg/ml) or PBS (50 ul) were injected intradermally into the pre-shaved back skin. After 20 minutes, the animals were euthanized and an area of skin that included the blue spot resulting from leakage of the dye was removed. Evan's blue dye was extracted from the skin by incubation with formamide for 5 days at room temperature, and the absorbance of extracted dye was measured at 620 nm using a spectrophotometer. The unpaired Student t test was used for statistical analysis.

Tissue Processing for Electron Microscopy

SCID mice were treated for three days with TNP-470 (30 mg/kg) or saline, following which anionic Ferritin tracer was injected i.v. into mice and 50 μl of human VEGF₁₆₅ (1 ng/μl), or 50 μl of PBS were injected intradermally into pre-shaved flank skin. After 15 min, animals were euthanized by cervical dislocation. Skin test and control sites were excised and fixed by immersion for 4 hours in freshly prepared 2.0% paraformaldehyde-2.5% glutaraldehyde-0.025% calcium chloride in 0.1 M sodium cacodylate buffer, pH 7.4. Tissues were postfixed for 2 h in 1.5% sym-collidine-buffered osmium tetroxide, stained en bloc with uranyl acetate, dehydrated in a graded series of alcohols, and embedded in Spurr resin as previously described (Dvorak et al., 1996; Feng et al., 1996). Thin sections were then cut, collected on carbon-Formvar-coated single slot grids, and viewed in an electron microscope (CM10; Philips, Eindhoven, The Netherlands).

Induction of Delayed-Type Hypersensitivity Reactions

Delayed-type hypersensitivity (DTH) reactions were induced in the skin of 8-week-old C57B1/6J male mice (n=5) as previously described (Dvorak et al., 1984). In order to induce an immune response to oxazolone, mice were first sensitized by topical application of 2% oxazolone solution in vehicle, acetone: olive oil (4:1 vol/vol), to the shaved abdomen (50 μl) and to each paw (5 μl). Mice were treated with TNP-470 (30 mg/kg s.c.) for three days prior to the second challenge with oxazolone. Five days after sensitization, the right ears were challenged by topical application of 10 μl of a 1% oxazolone solution; the left ears were treated with vehicle alone. Ear thickness was then measured daily for up to 7 days as a measure of inflammation intensity (Gad et al., 1986). Statistical analysis was performed using the unpaired Student t test. Some mice from each experimental group were euthanized 24 hours after oxazolone challenge (n=5 per group). One half of each ear was fixed in 10% formalin and was processed, embedded in paraffin and stained for H & E. The other half was embedded in OCT compound (Sakura Finetek, Torrance, Calif.) and snap-frozen in liquid nitrogen. Immunohistochemical staining was performed on 5 μm cryostat sections using a Vecstatin avidin-biotin detection system (Vector Labs, Burlingame, Calif.) with rat monoclonal antibodies against mouse CD31 (dilution 1:250, Pharmingen, San Diego, Calif.) according to the manufacturers' instructions.

IL-2 Associated Pulmonary Edema

C57B1/6J male mice were injected with TNP-470 (30 mg/kg daily) or saline subcutaneously (s.c.) for three days. Then mice were injected with IL-2 (1.2×106 units/100 μl) or saline intraperitoneally (i.p.) 3 times a day for 5 days. At termination mice were euthanized and lungs were dissected, weighed, fixed and processed for H & E staining.

Miles Assay on Tumor-Bearing Mice

Female SCID mice (˜8 weeks, ˜20 g) were inoculated s.c. with 5×10⁶ viable U87 glioblastoma cells or viable A2058 human melanoma cells or BXPC3 pancreas adenocarcinoma cells. Female nu/nu mice were inoculated with MCF-7 or MDA-MB-231 breast carcinoma cells in the mammary fat pad. C57B1/6J were inoculated with LLC. When tumors reached a volume of approximately 100 mm3, mice were injected s.c. with free TNP-470 or HPMA copolymer-TNP-470 (30 mg/Kg TNP-equiv.) for three days, angiostatin (200 mg/kg s.c.) for five days, or saline (250 μl s.c.) for five days (n=10). Evan'sEvan's blue dye was then injected i.v. and extravasation of dye assessed as above. Also, blood was withdrawn to measure VPF/VEGF levels in plasma and in tumors (n=5 from each group). Tumors (n=5 per group) were dissected, weighed and cut in half. Half of the tumor was placed in formalin and half was analyzed for VPF/VEGF protein by ELISA (see below). Formalin fixed tumors were processed for sectioning and staining with H & E, CD31, smooth muscle actin (SMA) and proliferating cell nuclear antigen (PCNA) according to the manufacturers' instructions.

ELISA Assays for VPF/VEGF

Blood drawn from tumor-bearing mice was centrifuged and plasma was collected. Solid tumors were homogenized and resuspended in lysis buffer. In addition, tumor cells were plated at 500,000 cells per well (six-well plates) and conditioned media from cells was collected 48 hours later. Levels of VPF/VEGF in plasma, tumors and culture supernatants were determined in duplicate samples by ELISA (R&D Systems, MN) according to the manufacturer's instructions. The limit of sensitivity of the assay was 15 pg/ml.

Cell Proliferation Assay

HMVEC-d cells were trypsinized (0.05% trypsin) and resuspended (15,000 cells/ml) in EBM-2 supplemented with 5% fetal bovine serum (FBS), plated onto gelatinized 24-well culture plates (0.5 ml/well), and incubated for 24 hours (37° C., 5% CO₂). The media was replaced with 0.5 ml of complete media (serum and growth factors; EGM-2 MV), and test substances were applied. Cells were challenged with free or conjugated TNP-470 (0.01 pg/ml to 1 mg/ml TNP-470-equivalent concentration). Control cells were grown with or without growth factors. U87 glioblastoma cells were washed with PBS, trypsinized and resuspended (5,000 cells/ml) in DMEM supplemented with 10% FBS, plated onto 24-well culture plates (0.5 ml/well), and incubated for 24 hours (37° C., 5% CO₂). The media was replaced with 0.5 ml of DMEM and 10% FBS, and the test sample applied. Cells were challenged with free or conjugated TNP-470 (0.01 pg/ml to 1 mg/ml TNP-470-equivalent concentration). Control cells were grown with or without 10% FBS. Both cell types were incubated for 72 hours, followed by trypsinization, resuspension in Hematall (Fisher Scientific, Pittsburgh, Pa.), and counted in a Coulter counter.

Cell Migration Assay

The motility response of HMVEC-d cells was assayed using a modified Boyden chamber. Cells were plated in T75-cm² flasks at 0.5×106 cells per flask and allowed to grow for 48 hours (˜70% confluent) prior to the migration assay. To facilitate cell adhesion, the upper membrane of a transwell (8 μm pore; Costar) was coated with fibronectin (10 μg/ml; Becton Dickinson) overnight at 4° C. Coated membranes were rinsed with PBS and allowed to air dry immediately before use. Cells were detached by trypsinization, treated with trypsinization neutralization solution (Clonetics), and resuspended at a final concentration of 5×10⁶ cells/ml in serum-free EBM-2 containing 0.1% BSA or free or conjugated TNP470 at equivalent concentrations of 1 ng/ml. Cells (50,000 in 100 μl) were added to the upper chamber of the transwell. Following a 2 hours incubation, EBM-2 or EBM-2 supplemented with VPF/VEGF (5 ng/ml) was added to the lower chamber and cells were allowed to migrate toward the bottom chamber for 4 hours in a humidified incubator containing 5% CO₂. Transwell filters were rinsed once with PBS and fixed and stained using a Diff-Quik staining kit (Baxter) following the manufacturer's protocol. Non-migrated cells were removed from the upper chamber with a cotton swab. Stained filters were cut out of the chamber and mounted onto slides using Permount (Fisher). The number of migrated cells was measured using microscopy (three fields from each membrane were captured using a 10× objective), and images were captured with a CCD camera using SPOT software. Total migration per membrane was quantified from the captured images using Scion Image software (National Institutes of Health). All experiments were run in triplicate.

VEGFR-2 Phosphorylation

Serum-starved (0.1% FBS in EBM-2 media for 24 hours) HMVEC-d or HUVEC were treated with 5 ng/ml TNP-470 and HPMA copolymer-TNP-470 at 37° C. for 2 hours, and then stimulated with 10 ng/ml of VEGF for 5 minutes. Stimulation was stopped by adding cold PBS. Cells were lysed with cold precipitation buffer (20 mM Tris-HCl, pH 7.5, 0.15 M NaCl, 1% Triton X-100, 1 mM PMSF, 1 mM Na3VO4, 1 mM EGTA, 1 μg/ml leupeptin, 0.5% aprotinin, and 2 μg/ml pepstatin A). 500 μg of lysate protein was incubated with 1 μg of antibody against VEGFR-2 for 2 hours, then with 50 μl of protein A-conjugated agarose-beads at 4° C. for 34 hours. After washing the beads with precipitation buffer, immunoprecipitates were resuspended in 2× SDS sample buffer for Western blot analysis with an antibody against phosphorylated tyrosine (pTyr).

RhoA Activation Assay

pGST-TRBD bacteria were grown and induced with isopropyl-thiogalactoside. The bacterial suspensions were divided into 50 ml aliquots and then harvested and frozen at −80° C. To prepare the GST-TRBD beads, each aliquot of frozen bacteria was resuspended in 2 ml of cold PBS, and then 20 μl of 1 M dithiothreitol (DTT), 20 μl of 0.2 M PMSF, and 40 μl of 50 mg/ml lysozyme were added. The sample was incubated on ice for 30 minutes. Next, 225 μL of 10% Triton X-100, 22.5 μL of IM MgCl₂, 22.5 μl of 2000 KU/ml DNAse were added and the sample was incubated on ice for another 30 min. The supernatant was collected and incubated with 100 μl glutathione-coupled Sepharose 4B beads (Pharmacia Biotech) at 4° C. for 45 minutes. The beads were then washed with bead washing buffer (PBS with 10 mM DTT and 1% Triton X-100) and resuspended in the same buffer to give a 50% bead slurry.

Meanwhile, 24 hours serum-starved HMVEC-d cells were treated with 5 ng/ml TNP-470 at 37° C. for 2 hours, and then stimulated with 10 ng/ml of VEGF, PAF (10 nM), or histamine (100 mM) for 5 minutes. Stimulation was stopped by adding cold PBS. Cells were lysed with lysis buffer (150 mM NaCl, 0.8 mM MgCl₂, 5 mM EGTA, 1% IGEPAL, 50 mM HEPES, pH 7.5, 1 mM PMSF, 10 μg/ml leupeptin, and 10 μg/ml aprotinin). The supernatant was isolated and incubated with 50 μl of GST-TRBD beads at 4° C. for 45 minutes. Protein bound to beads was washed with AP wash buffer (50 mM Tris-HCl, pH 7.2, 1% Triton X-100, 150 mM NaCl, 10 mM MgCl₂, 1 mM PMSF, 10 μg/ml leupeptin, and 10 μg/ml aprotinin) and analyzed by SDS-PAGE with an antibody against RhoA (Santa Cruz Biotechnology, CA).

Phosphorylation of MAPK

Serum-starved HMVEC-d were treated with 5 ng/ml TNP-470 at 37° C. for 2 hours, and then stimulated with 10 ng/ml of VEGF for 5 minutes. Stimulation was stopped by adding cold PBS. Cells were lysed with cold radioimmune precipitation buffer. Cellular extracts (20 μg) were immunoblotted with an antibody against phosphorylated MAPK (p-MAPK) (Cell Signaling Technology Inc.). The blot was stripped and reprobed with an antibody against MAPK to confirm equal protein loading.

Intracellular Ca²⁺ Release

For detaching cells, serum-starved HMVEC-d were incubated with 4 ml of collagenase solution (0.2 mg/ml collagenase, 0.2 mg/ml soybean trypsin inhibitor, 1 mg/ml BSA, and 2 mM EDTA in PBS) at 37° C. for 30 minutes. Cell pellets were washed with 2 ml of Ca²⁺ buffer (5 mM KCl, 140 mM NaCl, 1 mM CaCl₂, 1 mM MgCl₂, 5.6 mM glucose, 0.1% BSA, 0.25 mM sulfinpyrazone, and 10 mM HEPEs, pH 7.5) and then resuspended in 2 ml of the same buffer containing 5 ng/ml TNP-470. The cells were incubated at 37° C. for 2 hours in suspension. During the last 30 minutes of incubation, 1 μg/ml Fura-2-AM Fura 2-AM (acetoxymethyl ester derivative of Fura 2) and 0.02% pluronic F-127 were added to the suspension. Cells (106) were collected and resuspended in 2 ml Ca²⁺ buffer for VPF/VEGF (10 ng/ml), PAF (20 nM), or histamine (100 mM) stimulation. Intracellular Ca²⁺ concentrations were measured with the DeltaScan Illumination System (Photon Technology International Inc.) using Felix software, while rocking the tubes.

Results

TNP-470, HPMA Copolymer-TNP-470 and Angiostatin Reduce Microvessel Permeability

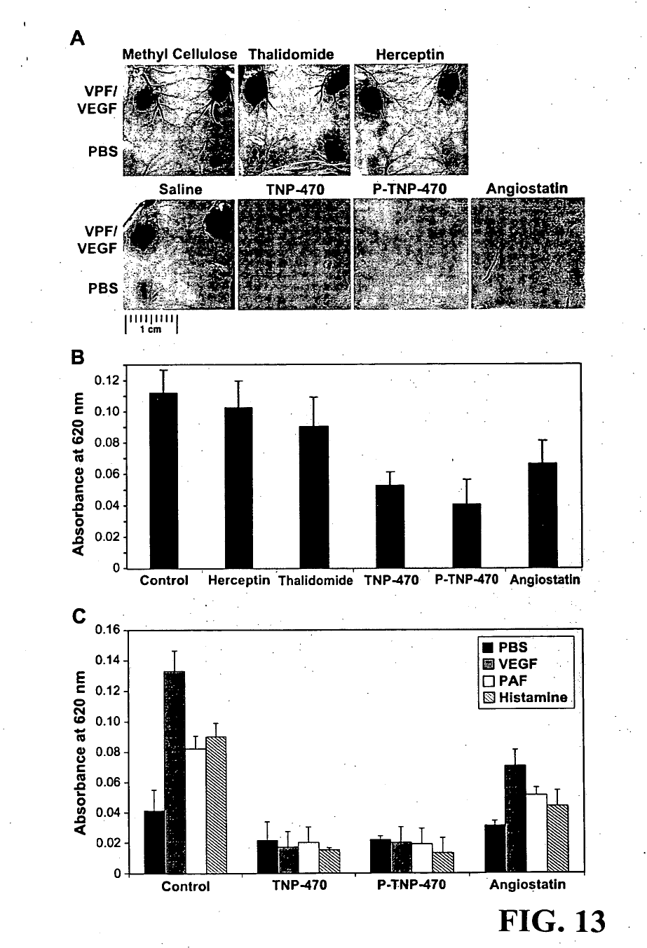

Vascular hyperpermeability is a prominent early feature of pathological angiogenesis. We first examined the effects of angiogenesis inhibitors that act directly on endothelial cells, such as TNP-470, HPMA copolymer-TNP-470 and angiostatin, on blood vessel permeability using the Miles assay (FIGS. 13A-13C). Evan's blue dye was injected i.v. followed by intradermal injection of VPF/VEGF, PAF, histamine and PBS into separate areas of shaved flank skin. Evan's blue dye binds to albumin and therefore extravasates along with albumin only at sites of increased permeability (Miles and Miles, 1952). Both free TNP-470 and HPMA copolymer-TNP-470 strikingly (˜70%) inhibited Evan's blue dye extravasation from VPF/VEGF injection sites (FIGS. 13A and 13B). This inhibitory effect required at least 24 hours pretreatment with TNP-470 or HPMA copolymer-TNP-470. Angiostatin also inhibited the vascular permeabilizing effects of VPF/VEGF but to a lesser extent (40%). Pretreatments with saline, Methyl Cellulose, Herceptin or thalidomide (both injected in methyl celulose) had no significant effect on VPF/VEGF-induced vessel permeability (FIGS. 13A and 13B). Herceptin and thalidomide are examples of angiogenesis inhibitors that act indirectly on endothelial cells (Kerbel and Folkman, 2002) by down-regulating expression of an oncogene by tumor cells (e.g., EGF receptor tyrosine kinase), blocking a product of that oncogene (e.g., VPF/VEGF), or blocking a receptor for that product (e.g., VEGFR, Anti-Erb B-2 monoclonal antibody). TNP-470 and HPMA-TNP-470 also decreased PAF- and histamine-induced permeability by 75 and 80% respectively (FIG. 13C). Pretreatment with angiostatin for 5 days reduced PAF- and histamine-induced permeability by 37% and 51% respectively (FIG. 13C). TNP-470 and HPMA copolymer-TNP-470 also blocked the low-level permeability induced by intradermal injections of PBS. This suggests that TNP-470 also blocks permeability induced by endogenous stimulators of permeability locally secreted in the injection site, such as serotonin and histamine. These results indicate that direct angiogenesis inhibitors inhibit vascular leakage induced by mediators that are thought to act by different mechanisms and through different signaling pathways.

In order to test the effect of TNP-470 on blood flow we injected 100 μl 1% Evan's blue i.v. into TNP-470 treated (30 mg/kg/d s.c. for 3 days) and control (Saline 250 μl/d s.c) mice. Five minutes later we performed punch biopsies of flank skin from both sets of mice and extracted these with formamide at RT for 5 days and read the extracts at 620 nm. There was no significant difference between the two groups (0.004±00.0001 and 0.005±0.0002). The rationale was that at 5 min after iv injection of Evan's Blue there is very little extravasation of dye and the vast majority is contained within the blood vasculature, thus providing a measure of blood volume and flow. If TNP-470 affected skin blood flow, for example by constricting blood vessels, we would have expected a reduction in Evan's blue dye.

Decreased Inflammation in TNP-470 Treated Mice

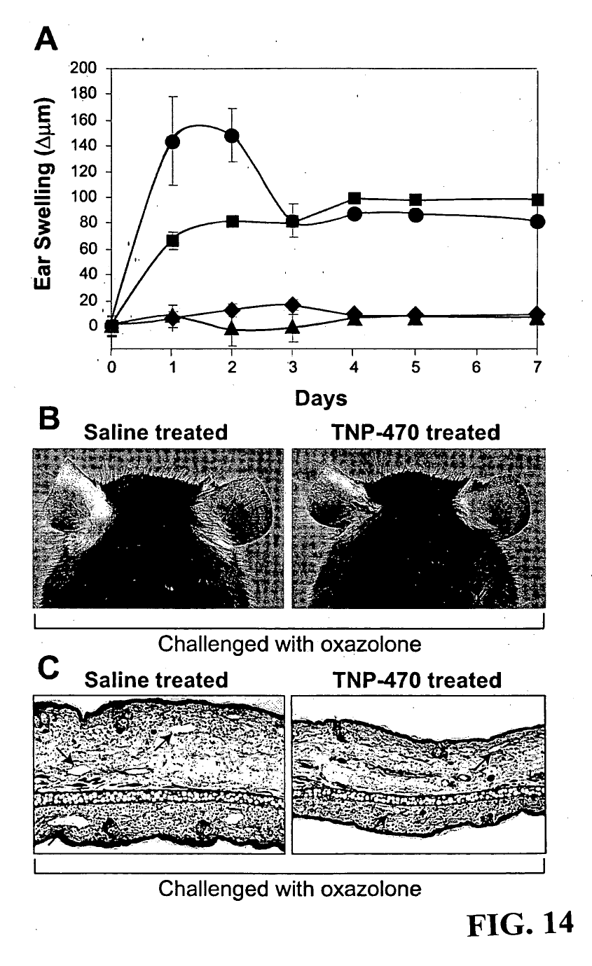

We sought to determine whether TNP-470's inhibitory effect on mediator-induced vessel permeability could be generalized to inflammation where vessels are hyperpermeable (Colvin and Dvorak, 1975). Delayed-type hypersensitivity (DTH) was induced with oxazolone in C57B1/6J mice that were treated with TNP-470 or saline as a control. Sensitized mice were then challenged with oxazolone and ear swelling was measured twenty-four hours later. TNP-470-treated mice had significantly reduced ear swelling and erythema as compared with control mice (P<0.01; FIGS. 14A and 14B). These differences persisted at 48 hours (P<0.01; FIG. 14A), but the differences had disappeared by 4 days as the inflammatory reaction subsided. No differences were found in the thickness of left ears that were sensitized with the vehicle (acetone olive oil), but not challenged with oxazolone (FIG. 14A).

Histology at 24 hours after challenge with oxazolone revealed typical delayed hypersensitivity reactions in the ears of sensitized mice that had been treated with saline rather than TNP-470. As expected, these ears showed extensive edema and accumulation of large numbers of lymphocytes and macrophages throughout the dermis that extended focally into the epidermis. In addition, the lymphatics were opened widely (arrows, FIG. 14C), a feature of increased lymphatic flow. In contrast, the inflammatory response was dramatically reduced in similarly sensitized and oxazolone-challenged mice that had been treated with TNP-470 (FIG. 14C). As expected from the ear thickness data (FIG. 14A), edema was greatly reduced as was the inflammatory cell infiltrate.

TNP-470 Decreases Pulmonary Edema Induced by IL-2

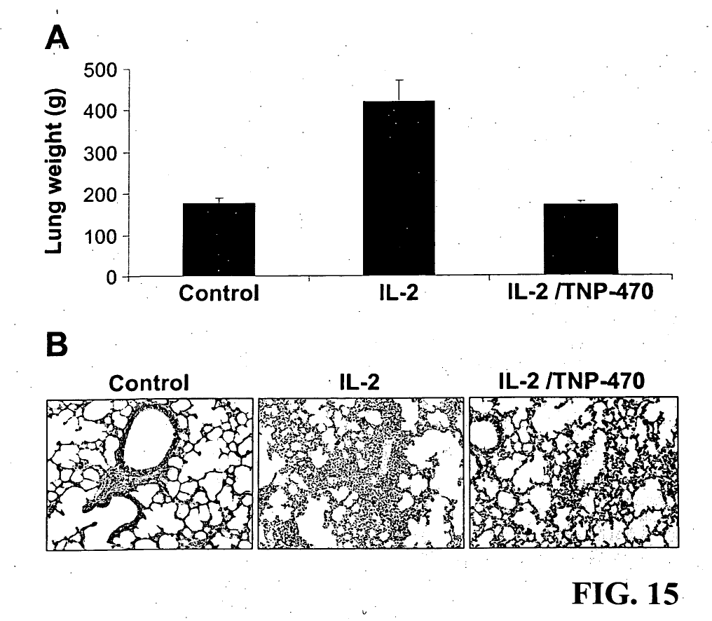

Patients with metastatic renal cell carcinoma have a generally poor prognosis, and there is currently no effective internationally recognized standard therapy for these patients. A subgroup of patients treated with interferon alpha or IL-2 monotherapy responds to these therapies, but almost every patient suffers from adverse side-effects. Treatment with IL-2 is known to produce widespread edema, a complication that has limited its use in the therapy of melanoma, metastatic renal cell carcinoma and other cancers (Ballmer-Weber et al., 1995; Berthiaume et al., 1995). Because of TNP-470's striking ability to limit vascular permeability and edema we determined whether treatment with TNP-470 could have a similar effect in an IL-2-induced pulmonary edema mouse model. As expected, IL-2-treated mice developed edematous lungs with wet weights of 2.5 times normal (419.4±50.4 mg); by contrast, the lungs of mice treated with both IL-2 and TNP-470 remained normal in weight (170.2±10.1 mg), similar to those of control animals (177.8±12.1 mg) that did not receive IL-2 (FIG. 15A). The short treatment (3 days only) with TNP-470 did not affect total body weight of mice, therefore the lower weight of the lungs of the mice treated with TNP-470 is not a result of general weight loss.

Histological examination of the lungs of IL-2 treated mice revealed severe congestion and edema with intra-alveolar fibrin deposition as well as a prominent mononuclear cell infiltrate that was predominantly peri-vascular and peri-bronchial. All of these pathological features were greatly reduced in TNP-470-treated mice (FIG. 15B).

TNP-470 Inhibits the Hyperpermeability of Tumor Blood Vessels

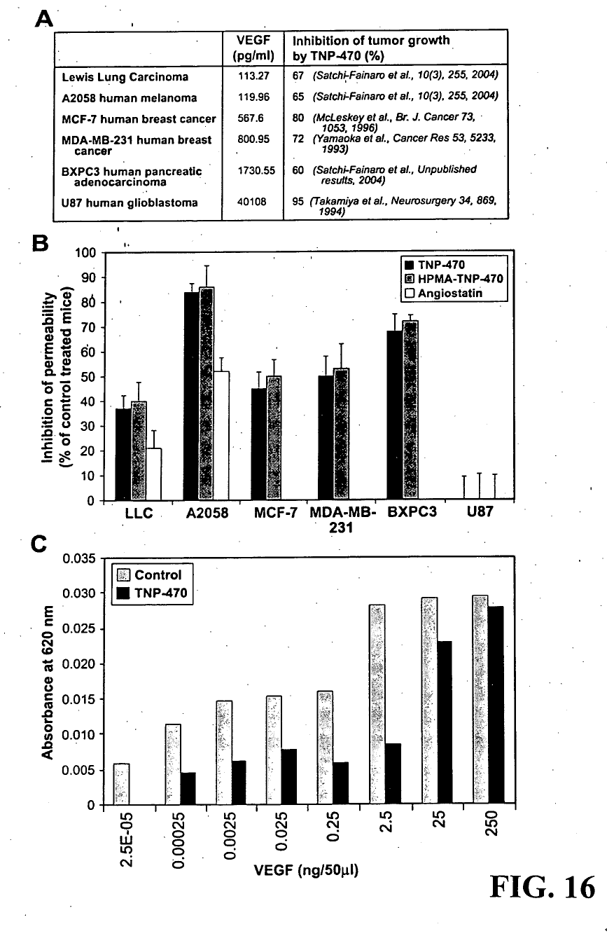

We examined six cell lines that differed widely in their expression of VPF/VEGF in vitro and in vivo. The two extremes were A2058 melanoma tumor line, which expressed negligible amounts of VPF/VEGF in culture, whereas the U87 glioblastoma cells secreted substantial amounts of VPF/VEGF into culture medium (FIG. 16A). Growth inhibition of these tumors by TNP-470 is summarized from the literature in the same table (FIG. 16A). All the tumor models studied were inhibited by 60-95% by TNP-470. Mice were implanted with these six tumor types and, when tumor size reached approximately 100 mm³, the mice were treated with TNP-470 or HPMA copolymer-TNP-470 conjugate for 3 days or with angiostatin for 5 days. Animals were then euthanized and tumors were dissected, homogenized and resuspended in lysis buffer. VPF/VEGF levels in A2058 melanoma tumors were measured at 20 pg/100 mg and in U87 glioblastomas at 3192±762 pg/100 mg.

TNP-470, HPMA copolymer-TNP-470 and angiostatin all inhibited Evan's blue extravasation into A2058 melanoma (P<0.03 versus control), murine Lewis lung carcinoma (P<0.05), MCF-7 breast carcinoma (P<0.04), MDA-MB-231 breast carcinoma (P<0.05) and BXPC3 pancreatic adenocarcinoma (P<0.04) by 40-90% compared to control tumors treated with saline (FIG. 16B). TNP-470, HPMA copolymer-TNP-470 and angiostatin did not inhibit Evan's blue extravasation into U87 glioblastomas (FIG. 16B). In order to test the effect of VEGF secreted from the tumor and the ability of TNP-470 to inhibit the permeability induced by such amount of VEGF, a modified Miles assay using a dose response of VEGF was used. Groups of 8 week old C57 mice were treated with TNP-470 (30 mg/kg/d s.c. for 3 days) or with saline. Then we injected 100 μl 1% Evan's Blue i.v. and after 10 min injected VEGF intradermally at different concentrations. We collected skin punch biopsies and extracted dye with formamide at RT over 5 days and read the extracts at 620 nm (FIG. 16C). Control saline-treated mice showed a dose-response correlation between increasing VEGF injection and dye accumulation, up to saturation. TNP-470-treated mice showed inhibition of permeability up to 25 ng but, above that dose, TNP-470 lost its effectiveness in inhibiting permeability and dye accumulation.

Following treatment with free or conjugated TNP-470 (for 3 days) or angiostatin (for 5 days), there was no significant difference in vessel density as determined by immunohistochemical staining for smooth muscle actin (SMA), proliferating cell nuclear antigen (PCNA) or CD31 staining in A2058 melanoma or U87 glioblastoma tumor models. CD31 staining of TNP-470, HPMA-TNP-470, angiostatin and untreated mice showed no difference in microvessel density in U87 glioblastoma (108±24, 120±13, 105±15, 118±30 microvessels per square mm ±standard error respectively). Differences in permeability cannot therefore be attributed to changes in vessel number or vessel density. Thus, for the first time we have shown an effect of TNP-470 on VEGF-induced permeability without an effect on vessel number or density (vascular proliferation).

TNP-470 does not Affect the Structure of Vesiculo-Vacuolar Organelles or of Inter-Endothelial Junctions

The vesiculo-vacuolar organelle (VVO) is a recently described structure in the endothelium of normal venules and of some tumor vessels (Feng et al., 1996). VVOs provide a major pathway for macromolecule extravasation when vascular permeability is increased by mediators such as VPF/VEGF, serotonin, and histamine. Ultrastructural enzyme-affinity cytochemistry and immunocytochemistry have localized VEGFR-2 to VVOs in vivo in mice (Feng et al., 1996).

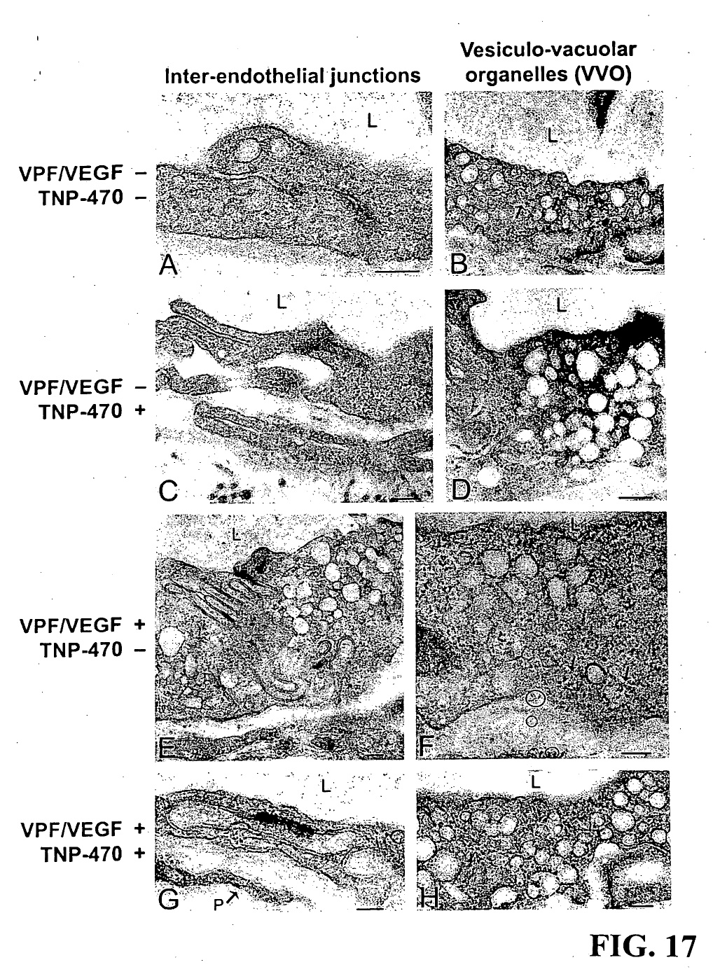

TNP-470 had no effect on the structure of VVOs or of inter-endothelial junctions in normal mouse skin or in skin injected with buffer (FIGS. 17A and 17B compared to 17C and 17D, respectively). Nevertheless, the minor extravasation of circulating ferritin via VVOs in uninjected skin or in skin injected with buffer (FIG. 17B) was reduced in mice treated with TNP-470 (FIG. 17D). Venule endothelial cells in mice injected locally with VPF/VEGF and systemically with buffer (FIGS. 17E and 17F) or with TNP-470 (FIGS. 17G and 17H) exhibited normally closed inter-endothelial cell junctions (FIGS. 17E and 17G) and structurally normal VVOs (FIGS. 17F and 17H). However, circulating ferritin extravasated through VVO vesicles at sites of VPF/VEGF injection and did so to a lesser extent in TNP-470-treated mice (FIGS. 17F and 17H). The anti-permeability effect of TNP-470 thus appears to be functional and not structural.

BCE cells were grown on a coverslip glass in a 24-well plate (200,000 cells/well) in DMEM+10% BCS+3 ng/ml bFGF. Cells were treated with TNP-470 for 3 days in culture. Cells were stimulated with VEGF (5 ng/ml) or PAF (100 nM) for 20 min. Cells were stained for occludin, claudin, ZO-1, beta-catenin and VE-cadherin with fluorescent antibodies. We did not see any significant and reproducible effect on occludin, claudin, ZO-1, beta-catenin or VE-cadherin on BCE cells in vitro while treated with TNP-470 and stimulated by VEGF or PAF as quantified by measuring fluorescence and evaluating differences in localization (Data not shown).

TNP-470 Inhibits VPF/VEGF-Induced VEGFR-2 Phosphorylation

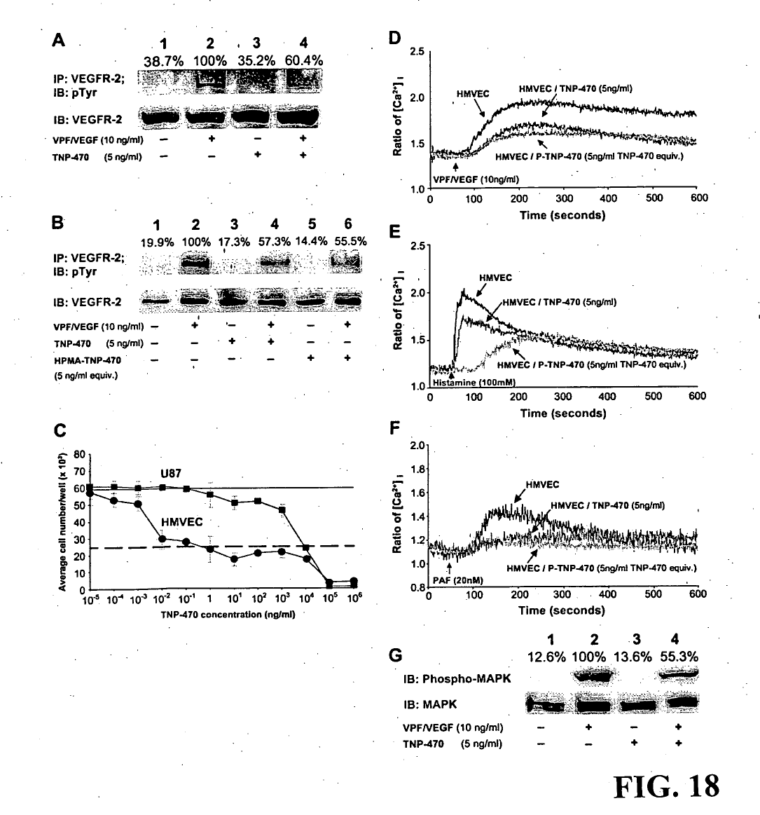

VPF/VEGF is thought to achieve its multiple effects on vascular endothelium primarily by activating VEGFR-2. Therefore, in order to investigate the molecular mechanisms of TNP-470 action, we investigated its effect on the VEGFR-2 signaling pathway. Incubation of HMVEC-d for 2 hours with 5 ng/ml TNP-470 or HUVEC for 2 hours with 5 ng/ml TNP-470 or HPMA copolymer-TNP-470 significantly reduced VPF/VEGF-induced phosphorylation of VEGFR-2 (HMVEC-d FIG. 18A, HUVEC FIG. 18B). We next investigated TNP-470 activities downstream of VEGFR-2 phosphorylation, such as endothelial cell proliferation and migration: Rho A activation (essential for VPF/VEGF-induced migration of endothelial cells), and calcium influx and MAPK activation (both essential for VPF/VEGF-induced endothelial cell proliferation (Zeng et al., 2001)).

TNP-470 Selectively Inhibits Endothelial Cell Proliferation, Ca2+ Influx and MAPK

We tested the effect of TNP-470 on endothelial and tumor cell proliferation in cultured HMVEC-d and U87 glioblastoma. TNP-470 inhibited growth factor-induced proliferation of HMVEC-d at concentrations as low as 1 pg/ml without causing cytotoxicity; only at concentrations higher than 1 μg/ml, did TNP-470 become cytotoxic (below the basal cell proliferation in the absence of growth factors in the media). TNP-470 inhibited serum-induced proliferation (cytostatic effect) of U87 glioblastoma cells but only at concentrations higher than 10 ng/ml (FIG. 18C) and was only cytotoxic to tumor cells at concentrations higher than 100 μg/ml. Thus, TNP-470 inhibited VPF/VEGF-induced endothelial cell proliferation at concentrations 4-orders of magnitude below that required to inhibit tumor cell growth. This difference in sensitivity between tumor and endothelial cell has been intensively investigated previously with different cell lines (Milkowski and Weiss, 1999; Satchi-Fainaro et al., 2004). HPMA copolymer-TNP-470 conjugate displayed a similar in vitro pattern of activity as unconjugated TNP-470 (Satchi-Fainaro et al., 2004). HPMA copolymer alone (without TNP-470) was inert in vitro and in vivo (data not shown).

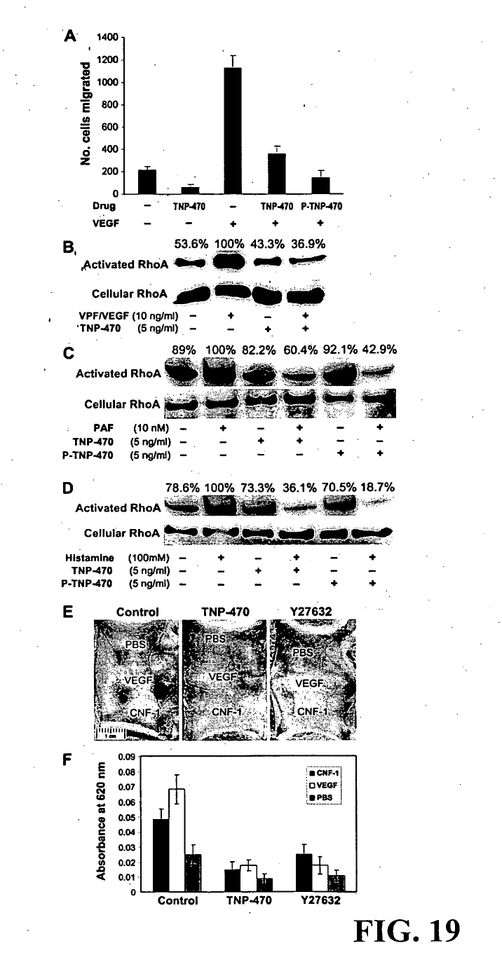

Increased endothelial cell calcium influx [Ca²⁺ ]i and MAPK activation are essential downstream steps in the VEGFR-2 signaling pathway that lead to endothelial cell proliferation (McLaughlin and De Vries, 2001). Increased [Ca 2+]i is also necessary for VPF/VEGF-mediated vascular permeability (Pal et al., 2000) (Mukhopadhyay and Dvorak, unpublished observations). TNP-470 and HPMA copolymer-TNP-470 conjugate decreased Ca²⁺ influx induced by VPF/VEGF (FIG. 18D), by histamine (FIG. 18E) and by PAF (FIG. 18F). TNP-470 treatment also inhibited VPF/VEGF-induced MAPK phosphorylation (FIG. 19G).

TNP-470 Inhibits Endothelial Cell Migration and RhoA Activation

We next examined the effect of TNP-470 on VPF/VEGF-induced endothelial cell migration through fibronectin-coated porous membranes in Transwell chambers. Migration was assessed by counting the number of cells that migrated through the membranes toward the chemoattractant during a 4 hours period following 2 hours pretreatment with free or conjugated TNP-470 (1 ng/ml). Treatment with TNP-470 or HPMA copolymer-TNP-470 dramatically inhibited the chemotactic migration response to VPF/VEGF by 68% (P=0.00045) and 87% (P=0.000096), respectively (FIG. 19A). In contrast, cells treated with HPMA copolymer alone (at 1 μg/ml TNP-470 equivalent concentration) migrated similarly to untreated control HMVEC-d. TNP-470 also inhibited basal migration of HMVEC-d cells in the absence of VPF/VEGF by 70% (P=0.0023).

The RhoA superfamily of small GTPase has been shown to play a key role in cell proliferation, shape change, and migration (Aspenstrom, 1999). RhoA and RacI are required for VEGFR-2-mediated HMVEC-d migration (Zeng et al., 2002). Therefore, we examined the possible role of RhoA in TNP-470's inhibition of VPF/VEGF-mediated HMVEC-d migration. VPF/VEGF-induced RhoA activation in HMVEC-d cells and this was significantly suppressed by TNP-470 (FIG. 19B). These results suggest that RhoA inhibition by TNP-470 at least in part leads to the inhibition of VPF/VEGF-induced migration of HMVEC-d. We also tested the effect of TNP-470 on PAF (FIG. 19C) and histamine (FIG. 19D)-induced RhoA activation and these were inhibited by TNP-470 and HPMA-TNP-470 as well.

To determine whether VPF/VEGF-induced permeability was mediated by the RhoA pathway, we used Y27632 a pharmacological inhibitor of Rock, a downstream target of RhoA (Breslin and Yuan, 2004). Rock is a kinase that has been implicated in the formation of cell-cell junctions. Pretreatment of SCID mice with Y27632 inhibited VPF/VEGF- and Escherichia coli cytotoxic necrotizing factor-1 (CNF-1)-induced Evan's blue-albumin extravasation (FIG. 19E). Activation of RhoA (along with Rac and Cdc42) with CNF-1 (Hopkins et al., 2003) was sufficient to promote extravasation, because CNF-1 induced vessel leakage in the Miles assay when injected intradermally (FIGS. 19E and 19F). This response was inhibited by Y27632, thus RhoA pathway appeared to be a key mediator of VPF/VEGF-induced leakage. Pretreatment of SCID mice with TNP-470 also inhibited CNF-1 induced Evan's blue-albumin extravasation (FIGS. 19E and 19F). These results suggest that systemic in vivo inhibition of RhoA causes inhibition of VEGF-induced vessel leakiness and that TNP-470 inhibits vessel leakiness through inhibition of RhoA activation.

Discussion

Tumor growth beyond a minimal size requires the generation of new blood vessels. Tumors induce these vessels by secreting angiogenic cytokines of which VPF/VEGF is arguably the most important. The new blood vessels that tumors induce are structurally and functionally abnormal and fail to provide tumors with an adequate blood supply (Jain, 2003). As a result, tumors often exhibit substantial zones of necrosis and are more susceptible than most normal tissues to factors that further compromise vascular function. To take advantage of this aspect of tumor biology a growing number of agents has been identified that in one way or another impair tumor angiogenesis and therefore tumor growth. Although antiangiogenic factors have attracted much attention, the mechanism of action of many has remained elusive. Some antiangiogenic agents have well-defined targets, such as anti-VEGF antibodies (Mordenti et al., 1999); in other instances, the targets of agents, such as phosphorylation inhibitors are more generic (Kerbel and Folkman, 2002). However, for such inhibitors as endostatin, angiostain and TNP-470 little is known about their molecular targets or the steps in the angiogenic pathway at which they act. In order to extend our understanding of the various actions of such antiangiogenic molecules, we tested their effect on vascular permeability, a distinctive component of pathological angiogenesis. In the present study, we have demonstrated that TNP-470, HPMA copolymer-TNP-470 and angiostatin strongly inhibit vascular leakage.

In the Miles assay, treatment with TNP-470 for as little as 24 hours was sufficient to inhibit extravasation of Evan's blue dye induced by potent vascular permeabilizing agents, VPF/VEGF, histamine, PAF and by IL-2-induced inflammation. TNP-470 also inhibited vascular leakage from the vasculature of 5 out of 6 different tumors that secreted variable levels of VPF/VEGF. Short-term treatment (1-3 days) with TNP-470 acted in tumors without causing changes in vessel density. TNP-470 inhibited RhoA activation in vitro and in vivo. Therefore, we also tested by immunohistochemistry the effect of TNP-470 on inter-endothelial junction proteins in endothelial cell cultures, such as VE-cadherin, occludin, claudin and zonula occludin-1 (ZO-1) and there was no significant effect in vitro (data not shown). In vivo, there was no effect on inter-endothelial junctions as shown by electron microscopy; instead, TNP-470 apparently affected the function, but not the structure of venular endothelial cell VVOs (FIG. 17).

We and others have previously shown that TNP-470 and HPMA copolymer-TNP-470 significantly inhibited the growth of A2058 human melanoma in SCID mice and, LLC in C57B1/6J mice (Satchi-Fainaro et al., 2004), as well as several other tumors (FIG. 16B). We showed here that TNP-470 and HPMA copolymer-TNP470 also inhibited the vascular permeability studying these same tumors. Interestingly, the only tumor whose vascular permeability was not blocked by TNP-470 or by HPMA-TNP-470 or by angiostatin was U87, a tumor that secretes extremely large amounts of VEGF. This suggests a threshold of VEGF above which there may be a need for a long-term treatment in order to achieve the inhibition of permeability observed in other tumors in this study. Thus, we conclude that the U87 tumors make so much VEGF that TNP-470 at the dose used is unable to prevent vascular permeability. However, TNP-470 was effective in blocking permeability in all of the other tumors tested, all of which made lesser amounts of VEGF. It may be that treatment of the U87 tumor (and perhaps glioblastomas in patients) will require supplemental therapy with other agents that specifically target VEGF, e.g., anti-VEGF antibodies, although this particular U87 glioblastoma in mice was inhibited by 95% by TNP-470 (FIG. 16A). It has been previously shown that antiangiogenic therapy produce a morphologically and functionally “normalized” vascular network (Tong et al., 2004). The normalization process prunes immature vessels and improves the integrity and function of the remaining vasculature by enhancing the perivascular cell and basement membrane coverage (Tong et al., 2004). Therefore, TNP-470 can normalize the vasculature by decreasing the hyperpermeability and combination therapy with other anticancer agents can be synergistic.

It has been shown that hyperpermeability of tumor blood vessels contributes to tumor progression and that blockade of microvascular permeability by blocking eNOS may be exploited as a novel target for antitumor therapy (Gratton et al., 2003). Together, these findings suggest that the anti-permeability effects of angiogenesis inhibitors may be a separate mechanism preceding their antitumor activity. Inhibition of permeability is not the only mechanism of action of TNP-470 and U87 tumor is an exception to the rule of an effect on permeability.

We also asked whether there is a relationship between the mechanisms by which TNP-470 inhibits VEGF-induced mitogenesis, migration and permeability. In vitro, TNP-470 selectively inhibited both HMVEC-d proliferation and migration. The signaling pathways mediating both of these functions, as well as permeability, are initiated by VEGFR-2 phosphorylation. Brief pretreatment with TNP-470 decreased VEGFR-2 phosphorylation. Consequently, TNP-470 inhibited MAPK phosphorylation that is downstream of VEGFR-2. TNP-470 also reduced VPF/VEGF-induced RhoA activation, a signaling step with a key role in both endothelial cell proliferation and migration. Further, it has been suggested that RhoA activation triggers Ca²⁺ entry via intracellular store depletion, leading to endothelial permeability (Mehta et al., 2003). RhoA is a major player in cytoskeleton organization and in cellular tension generation (Hall, 1998; Ingber, 2002). Furthermore, it has been shown recently that endostatin, thrombospondin-1, fumagillin, and TNP-470 target the endothelial cell cytoskeleton through altered regulation of heat shock protein 27 and cofilin (Keezer et al., 2003). Western blotting and immunofluorescence experiments confirmed that the phosphorylation states and subcellular localization of these two proteins were affected by all of the inhibitors tested and that treated cells had a more extensive network of actin stress fibers and more numerous focal adhesion plaques than untreated cells (Keezer et al., 2003). This effect may further contribute to the inhibitory effect of TNP-470 and endostatin (S. Soker, personal communication) on vessel leakage.

We also showed that TNP-470 prevented pulmonary edema induced by IL-2. Therefore, we demonstrate that TNP-470, and particularly its non-toxic HPMA copolymer-TNP-470 conjugate, are useful for alleviating the pulmonary edema that limits the use of IL-2 in the treatment of patients with malignant melanoma, renal cell carcinoma and other tumors (Lotze et al., 1986; Topalian and Rosenberg, 1987).

Neovascularization in malignant gliomas is also responsible for peritumoral brain edema (Cox et al., 1976), which causes life-threatening events. Chronic high-dose corticosteroid therapy, the current standard treatment for peritumoural brain edema, is associated with serious adverse effects including muscle wasting, gastrointestinal bleeding, osteoporosis and central nervous system effects ranging from personality changes to frank psychoses. Furthermore, peritumoral brain edema may facilitate the spreading of glioma cells (Gabbert, 1985; Ohnishi et al., 1990). Thus, it is possible that inhibition of tumor angiogenesis controls not only tumor growth but also glioma invasion by blocking vessel permeability.

VPF/VEGF is a multifunctional cytokine secreted by tumor cells and is thought to be responsible for the hyperpermeable state of tumor blood vessels (Carmeliet and Collen, 2000; Dvorak et al., 1991; Matsumoto and Claesson-Welsh, 2001). Increased tumor vessel permeability contributes to the extravascular deposition of plasma proteins and the fibrin gel that provide a provisional matrix that favors the migration of fibroblasts and endothelial cells into tumors. Angiogenesis inhibitors affect differently tumor vessel permeability in diverse tumors (FIG. 16B). Here we show six different tumors that were inhibited by free or conjugated TNP-470 (Table 16A), but their vessel permeability was diversely affected. It appears that there is a limit of VPF/VEGF expression in tumors above which TNP-470 does not detectably reduce vascular permeability. TNP-470 therefore may limit the growth of such tumors by other mechanisms.

VPF/VEGF stimulates transient accumulation of cytoplasmic calcium in cultured endothelial cells (Brock et al., 1991). The VPF/VEGF, PAF or histamine-induced increase in endothelial cytosolic Ca²⁺ likely activates calcium-calmodulin-dependent enzymes such as endothelial constitutive nitric oxide synthase. Nitric oxide has been implicated in the VPF/VEGF driven vascular leakiness (Fukumura et al., 2001; Murohara et al., 1998). Ku et al. (Ku et al., 1993) previously showed that VPF/VEGF stimulates nitric oxide production in isolated canine coronary arteries. Murohara et al. extended these observations by demonstrating that VPF/VEGF also stimulates nitric oxide release from cells regulating vascular permeability at the microvascular level. Activation of endothelial nitric oxide synthase (eNOS) by VPF/VEGF involves several pathways including Akt/PKB, Ca²⁺/calmodulin, and protein kinase C (Aoyagi et al., 2003; Aramoto et al., 2004). Here, we have shown that VEGF-dependent and VEGF-independent Ca²⁺ influx is inhibited by TNP-470. Moreover, it has been previously shown that TNP-470 inhibits nitric oxide production (Mauriz et al., 2003; Yoshida et al., 1998). Therefore, vessel leakiness dependence on eNOS (Gratton et al., 2003), which is Ca²⁺ dependent, is inhibited. In addition to TNP-470's previously established ability to bind methionine amiopeptidase-2 (Griffith et al., 1997), we now show additional mechanisms of action for this drug. Together, inhibition of VEGFR-2 phosphorylation, RhoA activation and Ca²⁺ influx provide a novel mechanism for TNP-470's effect on proliferation, migration and now, vascular leakiness.

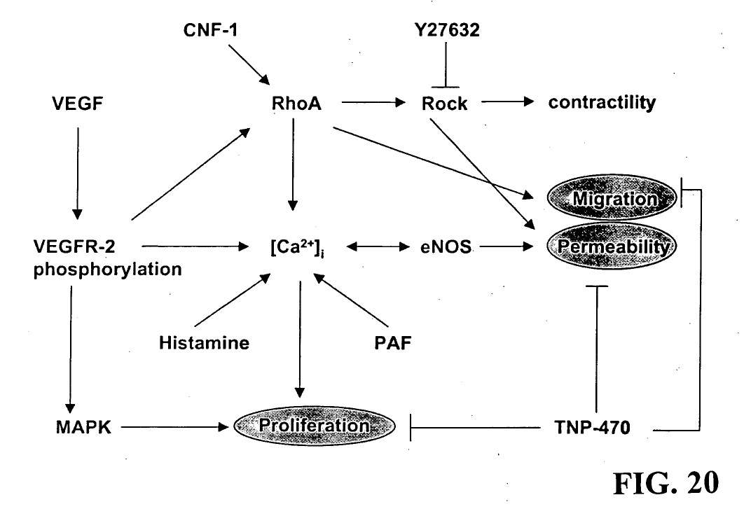

TNP-470 and HPMA copolymer-TNP-470 inhibited vascular leakiness induced by three different agonists (PAF, VPF/VEGF and histamine). Preliminary data has also shown that TNP-470 inhibits PAF synthesis (M. Sirois, personal communication). Our findings suggest that TNP-470 acts as an anti-permeability factor by inhibiting [Ca²⁺ ]i as proposed in our model (FIG. 20). Taken together, TNP-470 has a broader therapeutic spectrum that extends beyond tumor therapy. TNP-470, in its polymer-conjugated, non-toxic form, is useful for treating other disorders associated with vascular leakage and edema such as pulmonary edema, ascites and inflammation. Moreover, they are useful as adjuvants to IL-2 tumor therapy in order to avoid the pulmonary edema associated with this treatment. There are in addition many other clinical applications for a drug that inhibits vascular permeability, including the following: (i) “Reperfusion” syndromes following ischemic injury in brain and heart, transplantation of organs, and surgery for removal of large tumors in the pelvis where major vessels must be occluded temporarily; (ii) Cerebral edema associated with brain tumors, head injury or stroke; (iii) Lymphedema associated with axillary lymph node dissection following mastectomy; and (iv) Allergic reactions associated with edema.

Our study shows that several inhibitors of angiogenesis, TNP-470, its novel non-toxic polymeric conjugate HPMA copolymer-TNP-470 and angiostatin, reduce plasma macromolecule extravasation from the pathologically hyperpermeable vasculature supplying tumors and inflammatory sites, and also from blood vessels rendered hyperpermeable by three vascular permeabilizing mediators, VEGF, PAF and histamine. These inhibitors also reduced edema in tumors and pulmonary edema induced by IL-2 therapy and thus are useful as adjuvant therapy for tumors, inflammatory conditions, or complications of chemotherapy or immunotherapy. Our results describe a novel mechanism of action for TNP-470 and possibly other endogenous proteins with antiangiogenic activity.

In summary, we investigated the effects of TNP-470 on vascular permeability. TNP-470 and HPMA copolymer-TNP-470 inhibited the vascular hyperpermeability characteristic of tumor blood vessels as well as that induced in mouse skin by different mediators. Treatment for three days with TNP-470 or angiostatin was sufficient to reduce leakiness of tumor blood vessels, delayed-type hypersensitivity and pulmonary edema induced by IL-2. TNP-470 inhibited VPF/VEGF-induced phosphorylation of VEGFR-2, calcium influx and Rho A activation in cultured endothelial cells. These results have identified an important new activity of TNP-470, that of inhibiting vessel hyperpermeability. This activity contributes to TNP-470's antiangiogenic effect and indicates that HPMA copolymer-TNP-470 can be used in the treatment of cancer and inflammation.

### Example 3

### References

The references cited below for Example 3 are incorporated herein by reference.
Aiello, L. P., Avery, R. L., Arrigg, P. G., Keyt, B. A., Jampel, H. D., Shah, S. T., Pasquale, L. R., Thieme, H., Iwamoto, M. A., Park, J. E., and et al. (1994). Vascular endothelial growth factor in ocular fluid of patients with diabetic retinopathy and other retinal disorders. N Engl J Med 331, 1480-1487.
Aoyagi, M., Arvai, A. S., Tainer, J. A., and Getzoff, E. D. (2003). Structural basis for endothelial nitric oxide synthase binding to calmodulin. Embo J 22, 766-775.
Aramoto, H., Breslin, J. W., Pappas, P. J., Hobson, I. R., and Duran, W. N. (2004). Vascular endothelial growth factor stimulates differential signaling pathways in the in vivo microcirculation. Am J Physiol Heart Circ Physiol in press.
Aspenstrom, P. (1999). Effectors for the Rho GTPases. Curr Opin Cell Biol 11, 95-102. Ballmer-Weber, B. K., Dummer, R., Kung, E., Burg, G., and Ballmer, P. E. (1995). Interleukin 2-induced increase of vascular permeability without decrease of the intravascular albumin pool. Br J Cancer 71, 78-82.
Berthiaume, Y., Boiteau, P., Fick, G., Kloiber, R., Sinclair, G. D., Fong, C., Poon, M. C., and Lafreniere, R. (1995). Pulmonary edema during IL-2 therapy: combined effect of increased permeability and hydrostatic pressure. Am J Respir Crit Care Med 152, 329-335.
Bhargava, P., Marshall, J. L., Rizvi, N., Dahut, W., Yoe, J., Figuera, M., Phipps, K., Ong, V. S., Kato, A., and Hawkins, M. J. (1999). A Phase I and pharmacokinetic study of TNP-470 administered weekly to patients with advanced cancer. Clin Cancer Res 5, 1989-1995.
Breslin, J. W., and Yuan, S. Y. (2004). Involvement of RhoA and Rho kinase in neutrophil-stimulated endothelial hyperpermeability. Am J Physiol Heart Circ Physiol 286, H1057-1062.
Brock, T. A., Dvorak, H. F., and Senger, D. R. (1991). Tumor-secreted vascular permeability factor increases cytosolic Ca2+ and von Willebrand factor release in human endothelial cells. Am J Pathol 138, 213-221.
Carmeliet, P., and Collen, D. (2000). Molecular basis of angiogenesis. Role of VEGF and VE-cadherin. Ann N Y Acad Sci 902, 249-262; discussion 262-244.
Claesson-Welsh, L. (2003). Signal transduction by vascular endothelial growth factor receptors. Biochem Soc Trans 31, 20-24.
Claffey, K. P., Brown, L. F., del Aguila, L. F., Tognazzi, K., Yeo, K. T., Manseau, E. J., and Dvorak, H. F. (1996). Expression of vascular permeability factor/vascular endothelial growth factor by melanoma cells increases tumor growth, angiogenesis, and experimental metastasis. Cancer Res 56, 172-181.
Colvin, R. B., and Dvorak, H. F. (1975). Role of the clotting system in cell-mediated hypersensitivity. II. Kinetics of fibrinogen/fibrin accumulation and vascular permeability changes in tuberculin and cutaneous basophil hypersensitivity reactions. J Immunol 114, 377-387.
Cox, D. J., Pilkington, G. J., and Lantos, P. L. (1976). The fine structure of blood vessels in ethylnitrosourea-induced tumours of the rat nervous system: with special reference to the breakdown of the blood-brain barrier. Br J Exp Pathol 57, 419-430.
Dvorak, A. M., Kohn, S., Morgan, E. S., Fox, P., Nagy, J. A., and Dvorak, H. F. (1996). The vesiculo-vacuolar organelle (VVO): a distinct endothelial cell structure that provides a transcellular pathway for macromolecular extravasation. J Leukoc Biol 59, 100-115.
Dvorak, A. M., Lett-Brown, M. A., Thueson, D. O., Pyne, K., Raghuprasad, P. K., Galli, S. J., and Grant, J. A. (1984). Histamine-releasing activity (HRA). III. HRA induces human basophil histamine release by provoking noncytotoxic granule exocytosis. Clin Immunol Immunopathol 32, 142-150.
Dvorak, H. F. (2002). Vascular permeability factor/vascular endothelial growth factor: a critical cytokine in tumor angiogenesis and a potential target for diagnosis and therapy. J Clin Oncol 20, 4368-4380.
Dvorak, H. F., Brown, L. F., Detmar, M., and Dvorak, A. M. (1995). Vascular permeability factor/vascular endothelial growth factor, microvascular hyperpermeability, and angiogenesis. Am J Pathol 146, 1029-1039.
Dvorak, H. F., Sioussat, T. M., Brown, L. F., Berse, B., Nagy, J. A., Sotrel, A., Manseau, E. J., Van de Water, L., and Senger, D. R. (1991). Distribution of vascular permeability factor (vascular endothelial growth factor) in tumors: concentration in tumor blood vessels. J Exp Med 174, 1275-1278.
Feng, D., Nagy, J. A., Dvorak, A. M., and Dvorak, H. F. (2000). Different pathways of macromolecule extravasation from hyperpermeable tumor vessels. Microvasc Res 59, 24-37.
Feng, D., Nagy, J. A., Dvorak, H. F., and Dvorak, A. M. (2002). Ultrastructural studies define soluble macromolecular, particulate, and cellular transendothelial cell pathways in venules, lymphatic vessels, and tumor-associated microvessels in man and animals. Microsc Res Tech 57, 289-326.
Feng, D., Nagy, J. A., Hipp, J., Dvorak, H. F., and Dvorak, A. M. (1996). Vesiculo-vacuolar organelles and the regulation of venule permeability to macromolecules by vascular permeability factor, histamine, and serotonin. J Exp Med 183, 1981-1986.
Ferrara, N., and Henzel, W. J. (1989). Pituitary follicular cells secrete a novel heparin-binding growth factor specific for vascular endothelial cells. Biochem Biophys Res Commun 161, 851-858.
Folkman, J. (2004). Angiogenesis and blood supply. In Cell cycle and growth control; Biomolecular regulation and cancer, G. S. Stein, and A. B. Pardee, eds. (Hoboken, New Jersey, Wiley-Liss), pp. 333-367.
Folkman, J., and Kalluri, R. (2003). Tumor angiogenesis. In Cancer Medicine, D. W. Kufe, R. E. Pollock, R. R. Weichselbaum, R. C. J. Bast, T. S. Gansler, J. F. Holland, and E. I. Frei, eds. (Hamilton, Ontario, Canada, B. C. Decker Inc.), pp. 161-194.
Fukumura, D., Gohongi, T., Kadambi, A., Izumi, Y., Ang, J., Yun, C. O., Buerk, D. G., Huang, P. L., and Jain, R. K. (2001). Predominant role of endothelial nitric oxide synthase in vascular endothelial growth factor-induced angiogenesis and vascular permeability. Proc Natl Acad Sci U S A 98, 2604-2609.
Gabbert, H. (1985). Mechanisms of tumor invasion: evidence from in vivo observations. Cancer Metastasis Rev 4, 293-309.
Gad, S. C., Dunn, B. J., Dobbs, D. W., Reilly, C., and Walsh, R. D. (1986). Development and validation of an alternative dermal sensitization test: the mouse ear swelling test (MEST). Toxicol Appl Pharmacol 84, 93-114.
Gratton, J. P., Lin, M. I., Yu, J., Weiss, E. D., Jiang, Z. L., Fairchild, T. A., Iwakiri, Y., Groszmann, R., Claffey, K. P., Cheng, Y. C., and Sessa, W. C. (2003). Selective inhibition of tumor microvascular permeability by cavtratin blocks tumor progression in mice. Cancer Cell 4, 31-39.
Griffith, E. C., Su, Z., Niwayama, S., Ramsay, C. A., Chang, Y. H., and Liu, J. O. (1998). Molecular recognition of angiogenesis inhibitors fumagillin and ovalicin by methionine aminopeptidase 2. Proc Natl Acad Sci USA 95, 15183-15188.
Griffith, E. C., Su, Z., Turk, B. E., Chen, S., Chang, Y. H., Wu, Z., Biemann, K., and Liu, J. O. (1997). Methionine aminopeptidase (type 2) is the common target for angiogenesis inhibitors AGM-1470 and ovalicin. Chem Biol 4, 461-471.
Hall, A. (1998). Rho GTPases and the actin cytoskeleton. Science 279, 509-514.
Herbst, R. S., Madden, T. L., Tran, H. T., Blumenschein, G. R., Jr., Meyers, C. A., Seabrooke, L. F., Khuri, F. R., Puduvalli, V. K., Allgood, V., Fritsche, H. A., Jr., et al. (2002). Safety and pharmacokinetic effects of TNP-470, an angiogenesis inhibitor, combined with paclitaxel in patients with solid tumors: evidence for activity in non-small-cell lung cancer. J Clin Oncol 20, 4440-4447.
Hopkins, A. M., Walsh, S. V., Verkade, P., Boquet, P., and Nusrat, A. (2003). Constitutive activation of Rho proteins by CNF-1 influences tight junction structure and epithelial barrier function. J Cell Sci 116, 725-742.
Ingber, D., Fujita, T., Kishimoto, S., Sudo, K., Kanamaru, T., Brem, H., and Folkman, J. (1990). Synthetic analogues of fumagillin that inhibit angiogenesis and suppress tumour growth. Nature 348, 555-557.
Ingber, D. E. (2002). Mechanical signaling and the cellular response to extracellular matrix in angiogenesis and cardiovascular physiology. Circ Res 91, 877-887.
Jain, R. K. (2002). Tumor angiogenesis and accessibility: role of vascular endothelial growth factor. Semin Oncol 29, 3-9.
Jain, R. K. (2003). Molecular regulation of vessel maturation. Nat Med 9, 685-693. Keezer, S. M., Ivie, S. E., Krutzsch, H. C., Tandle, A., Libutti, S. K., and Roberts, D. D. (2003). Angiogenesis inhibitors target the endothelial cell cytoskeleton through altered regulation of heat shock protein 27 and cofilin. Cancer Res 63, 6405-6412.
Kerbel, R., and Folkman, J. (2002). Clinical translation of angiogenesis inhibitors. Nat Rev Cancer 2, 727-739.
Koch, A. E., Harlow, L. A., Haines, G. K., Amento, E. P., Unemori, E. N., Wong, W. L., Pope, R. M., and Ferrara, N. (1994). Vascular endothelial growth factor. A cytokine modulating endothelial function in rheumatoid arthritis. J Immunol 152, 4149-4156.
Kragh, M., Spang-Thomsen, M., and Kristjansen, P. E. (1999). Time until initiation of tumor growth is an effective measure of the anti-angiogenic effect of TNP-470 on human glioblastoma in nude mice. Oncol Rep 6, 759-762.
Ku, D. D., Zaleski, J. K., Liu, S., and Brock, T. A. (1993). Vascular endothelial growth factor induces EDRF-dependent relaxation in coronary arteries. Am J Physiol 265, H586-592.
Kusaka, M., Sudo, K., Matsutani, E., Kozai, Y., Marui, S., Fujita, T., Ingber, D., and Folkman, J. (1994). Cytostatic inhibition of endothelial cell growth by the angiogenesis inhibitor TNP-470 (AGM-1470). Br J Cancer 69, 212-216.
Lotze, M. T., Custer, M. C., and Rosenberg, S. A. (1986). Intraperitoneal administration of interleukin-2 in patients with cancer. Arch Surg 121, 1373-1379. Matsumoto, T., and Claesson-Welsh, L. (2001). VEGF receptor signal transduction. Sci STKE 2001, RE21.
Mauriz, J. L., Linares, P., Macias, R. I., Jorquera, F., Honrado, E., Olcoz, J. L., Gonzalez, P., and Gonzalez-Gallego, J. (2003). TNP-470 inhibits oxidative stress, nitric oxide production and nuclear factor kappa B activation in a rat model of hepatocellular carcinoma. Free Radic Res 37, 841-848.
McLaughlin, A. P., and De Vries, G. W. (2001). Role of PLCgamma and Ca(2+) in VEGF- and FGF-induced choroidal endothelial cell proliferation. Am J Physiol Cell Physiol 281, C1448-1456.
Mehta, D., Ahmmed, G. U., Paria, B., Holinstat, M., Voyno-Yasenetskaya, T., Tiruppathi, C., Minshall, R. D., and Malik, A. B. (2003). RhoA interaction with inositol 1,4,5-triphosphate receptor and transient receptor potential channel-1 regulates Ca2+ entry. Role in signaling increased endothelial permeability. J Biol Chem, M302401200.
Miles, A. A., and Miles, E. M. (1952). Vascular reactions to histamine, histamine liberators or leukotoxins in the skin of the guinea pig. J Physiol 118, 228-257.
Milkowski, D. M., and Weiss, R. A. (1999). TNP-470. In Antiangiogenic agents in cancer therapy, T. A. Teicher, ed. (Totowa, N.J., Human Press Inc.), pp. 385-398.
Mordenti, J., Thomsen, K., Licko, V., Chen, H., Meng, Y. G., and Ferrara, N. (1999). Efficacy and concentration-response of murine anti-VEGF monoclonal antibody in tumor-bearing mice and extrapolation to humans. Toxicol Pathol 27, 14-21.
Murohara, T., Asahara, T., Silver, M., Bauters, C., Masuda, H., Kalka, C., Kearney, M., Chen, D., Symes, J. F., Fishman, M. C., et al. (1998). Nitric oxide synthase modulates angiogenesis in response to tissue ischemia. J Clin Invest 101, 2567-2578.
Neufeld, G., Cohen, T., Gengrinovitch, S., and Poltorak, Z. (1999). Vascular endothelial growth factor (VEGF) and its receptors. Faseb J 13, 9-22.
Ohnishi, T., Sher, P. B., Posner, J. B., and Shapiro, W. R. (1990). Capillary permeability factor secreted by malignant brain tumor. Role in peritumoral brain edema and possible mechanism for anti-edema effect of glucocorticoids. J Neurosurg 72, 245-251.
O'Reilly, M. S., Holmgren, L., Shing, Y., Chen, C., Rosenthal, R. A., Moses, M., Lane, W. S., Cao, Y., Sage, E. H., and Folkman, J. (1994). Angiostatin: a novel angiogenesis inhibitor that mediates the suppression of metastases by a Lewis lung carcinoma. Cell 79, 315-328.
Pal, S., Iruela-Arispe, M. L., Harvey, V. S., Zeng, H., Nagy, J. A., Dvorak, H. F., and Mukhopadhyay, D. (2000). Retinoic acid selectively inhibits the vascular permeabilizing effect of VPF/VEGF, an early step in the angiogenic cascade. Microvasc Res 60, 112-120.
Ren, X. D., Kiosses, W. B., and Schwartz, M. A. (1999). Regulation of the small GTP-binding protein Rho by cell adhesion and the cytoskeleton. Embo J 18, 578-585.
Rihova, B., Bilej, M., Vetvicka, V., Ulbrich, K., Strohalm, J., Kopecek, J., and Duncan, R. (1989). Biocompatibility of N-(2-hydroxypropyl) methacrylamide copolymers containing adriamycin. Immunogenicity, and effect on haematopoietic stem cells in bone marrow in vivo and mouse splenocytes and human peripheral blood lymphocytes in vitro. Biomaterials 10, 335-342.
Satchi-Fainaro, R., Puder, M., Davies, J. W., Tran, H. T., Sampson, D. A., Greene, A. K., Corfas, G., and Folkman, J. (2004). Targeting angiogenesis with a conjugate of HPMA copolymer and TNP-470. Nat Med 10, 255-261.
Senger, D. R., Galli, S. J., Dvorak, A. M., Perruzzi, C. A., Harvey, V. S., and Dvorak, H. F. (1983). Tumor cells secrete a vascular permeability factor that promotes accumulation of ascites fluid. Science 219, 983-985.
Sin, N., Meng, L., Wang, M. Q., Wen, J. J., Bornmann, W. G., and Crews, C. M. (1997). The anti-angiogenic agent fumagillin covalently binds and inhibits the methionine aminopeptidase, MetAP-2. Proc Natl Acad Sci USA 94, 6099-6103.
Streit, M., Velasco, P., Riccardi, L., Spencer, L., Brown, L. F., Janes, L., Lange-Asschenfeldt, B., Yano, K., Hawighorst, T., Iruela-Arispe, L., and Detmar, M. (2000). Thrombospondin-1 suppresses wound healing and granulation tissue formation in the skin of transgenic mice. Embo J 19, 3272-3282.
Tong, R. T., Boucher, Y., Kozin, S. V., Winkler, F., Hicklin, D. J., and Jain, R. K. (2004). Vascular normalization by vascular endothelial growth factor receptor 2 blockade induces a pressure gradient across the vasculature and improves drug penetration in tumors. Cancer Res 64, 3731-3736.
Topalian, S. L., and Rosenberg, S. A. (1987). Therapy of cancer using the adoptive transfer of activated killer cells and interleukin-2. Acta Haematol 78 Suppl 1, 75-76. Yoshida, T., Kaneko, Y., Tsukamoto, A., Han, K., Ichinose, M., and Kimura, S. (1998). Suppression of hepatoma growth and angiogenesis by a fumagillin derivative TNP470: possible involvement of nitric oxide synthase. Cancer Res 58, 3751-3756.
Zeng, H., Dvorak, H. F., and Mukhopadhyay, D. (2001). Vascular permeability factor (VPF)/vascular endothelial growth factor (VEGF) peceptor-1 down-modulates VPF/VEGF receptor-2-mediated endothelial cell proliferation, but not migration, through phosphatidylinositol 3-kinase-dependent pathways. J Biol Chem 276, 26969-26979.
Zeng, H., Zhao, D., and Mukhopadhyay, D. (2002). KDR stimulates endothelial cell migration through heterotrimeric G protein Gq/11-mediated activation of a small GTPase RhoA. J Biol Chem 277, 46791-46798.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

### REFERENCES

The references cited below and incorporated throughout the application are incorporated herein by reference.
1. Ingber D et al, Nature 348, 555-557 (1990).
2. Kudelka et al., New Engl J Med 338, 991-992 (1998).
3. Folkman J., Angiogenesis, In Harrison's Textbook of Internal Medicine 15^{th} Edition, edited by E. Braunwald et al., McGraw Hill, pp. 517-530 (2001).
4. R. Duncan, J. K. Coatsworth, S. Burtles, Hum Exp Toxicol 17, 93-104 (1998).
5. H. Maeda, J. Wu, T. Sawa, Y. Matsumura, K. Hori, J Controlled Release 65, 271-84 (2000).
6. Claffey et al., Cancer Res 56, 172-181 (1996).
7. Andriopoulou, P., Navarro, P., Zanetti, A., Lampugnani, M. G., and Dejana, E. (1999). Histamine induces tyrosine phosphorylation of endothelial cell-to-cell adherens junctions., Arterioscler Thromb Vasc Biol 19, 2286-97.
8. Antonetti, D. A., Barber, A. J., Hollinger, L. A., Wolpert, E. B., and Gardner, T. W. (1999). Vascular endothelial growth factor induces rapid phosphorylation of tight junction proteins occludin and zonula occluden 1. A potential mechanism for vascular permeability in diabetic retinopathy and tumors., J Biol Chem 274, 23463-7.
9. Antonetti, D. A., Barber, A. J., Khin, S., Lieth, E., Tarbell, J. M., and Gardner, T. W. (1998). Vascular permeability in experimental diabetes is associated with reduced endothelial occludin content: vascular endothelial growth factor decreases occludin in retinal endothelial cells. Penn State Retina Research Group., Diabetes 47, 1953-9.
10 Archer, C. B. (1993). Platelet activating factor—a mediator of inflammation in the skin—medical implications., Clin Exp Dermatol 18, 489-95.
11. Bussolino, F., and Camussi, G. (1995). Platelet-activating factor produced by endothelial cells. A molecule with autocrine and paracrine properties., Eur J Biochem 229, 327-37.
12. Clough, G. F., Boutsiouki, P., and Church, M. K. (2001). Comparison of the effects of levocetirizine and loratadine on histamine-induced wheal, flare, and itch in human skin., Allergy 56, 985-8.
13. Connolly, D. T., Heuvelman, D. M., Nelson, R., Olander, J. V., Eppley, B. L., Delfino, J. J., Siegel, N. R., Leimgruber, R. M., and Feder, J. (1989a). Tumor vascular permeability factor stimulates endothelial cell growth and angiogenesis., J Clin Invest 84, 1470-8.
14. Connolly, D. T., Olander, J. V., Heuvelman, D., Nelson, R., Monsell, R., Siegel, N., Haymore, B. L., Leimgruber, R., and Feder, J. (1989b). Human vascular permeability factor. Isolation from U937 cells., J Biol Chem 264, 20017-24.
15. Dejana, E. (1997). Endothelial adherens junctions: implications in the control of vascular permeability and angiogenesis., J Clin Invest 100, S7-10.
16. Dvorak, H. F. (1986). Tumors: wounds that do not heal. Similarities between tumor stroma generation and wound healing., N Engl J Med 315, 1650-9.
17. Dvorak, H. F., Brown, L. F., Detmar, M., and Dvorak, A. M. (1995). Vascular permeability factor/vascular endothelial growth factor, microvascular hyperpermeability, and angiogenesis., Am J Pathol 146, 1029-39.
18. Esser, S., Wolburg, K., Wolburg, H., Breier, G., Kurzchalia, T., and Risau, W. (1998). Vascular endothelial growth factor induces endothelial fenestrations in vitro., J Cell Biol 140, 947-59.
19. Fischer, S., Clauss, M., Wiesnet, M., Renz, D., Schaper, W., and Karliczek, G. F. (1999). Hypoxia induces permeability in brain microvessel endothelial cells via VEGF and NO., Am J Physiol 276, C812-20.
20. Fischer, S., Wobben, M., Marti, H. H., Renz, D., and Schaper, W. (2002). Hypoxia-Induced Hyperpermeability in Brain Microvessel Endothelial Cells Involves VEGF-Mediated Changes in the Expression of Zonula Occludens-1., Microvasc Res 63, 70-80.
21. Martin, T. A. (2001). Tight junctions and their role in cancer metastasis., Histol Histopathol 16, 1183-95.
22. Murohara, T., Horowitz, J. R., Silver, M., Tsurumi, Y., Chen, D., Sullivan, A., and Isner, J. M. (1998). Vascular endothelial growth factor/vascular permeability factor enhances vascular permeability via nitric oxide and prostacyclin., Circulation 97, 99-107.
23. Roberts, W. G., and Palade, G. E. (1995). Increased microvascular permeability and endothelial fenestration induced by vascular endothelial growth factor., J Cell Sci 108, 2369-79.
24. Saria, A., and Lundberg, J. M. (1983). Evans blue fluorescence: quantitative and morphological evaluation of vascular permeability in animal tissues., J Neurosci Methods 8, 41-9.
25. Thurston, G., Suri, C., K, S., J, M., TN, S., GD, Y., and DM, M. (1999). Leakage-resistant blood vessels in mice transgenically overexpressing angiopoietin-1., Science 286,2511-4.
26. Wang, W., Dentler, W. L., and Borchardt, R. T. (2001). VEGF increases BMEC monolayer permeability by affecting occludin expression and tight junction assembly., Am J Physiol Heart Circ Physiol 280, H434-40.

1. A method of decreasing or inhibiting vascular hyperpermeability in an individual in need thereof, comprising administering to said individual an effective amount of compound selected from the group consisting of endostatin, thrombospondin, angiostatin, tumstatin, arrestin, recombinant EPO, and polymer conjugated TNP470.

2. The method of claim 1, wherein the vascular permeability is the result of a disease selected from the group consisting of non-proliferative diabetic retinopathy, diabetic nephropathy, nephrotic syndrome, pulmonary hypertension, allergic reactions associated with edema, lymphedema, cerebral edema, brain tumor edema, burn edema, tumor edema, reperfusion syndromes, and IL-2 therapy-associated edema.

3. A method of decreasing or inhibiting leakage from blood vessels of natural angiogenesis inhibitors in an individual in need thereof, comprising administering to said individual an effective amount of compound selected from the group consisting of endostatin, thrombospondin, angiostatin, tumstatin, arrestin, recombinant EPO, and polymer conjugated TNP-470.

4. A method of treating and/or preventing a non-proliferative diabetic retinopathy in an individual in need thereof comprising administering to said individual an effective amount of a compound selected from the group consisting of endostatin, thrombospondin, angiostatin, tumstatin, arrestin, recombinant EPO, and polymer conjugated TNP-470.

5. A method of decreasing or inhibiting vascular hyperpermeability in an individual in need of such treatment comprising administering to the individual an effective amount of a compound capable of stabilizing tight junction complexes.

6. The method of claim 5, wherein the compound capable of stabilizing tight junction proteins is selected from the group consisting of endostatin, thrombospondin, angiostatin, tumstatin, arrestin, recombinant EPO, and polymer conjugated TNP-470.

7. A method of screening for compounds that stabilize tight junction complexes comprising:
a) culturing endothelial cells in the presence of a test compound;
b) incubating said cultured endothelial cells expressing junction proteins; and
c) assessing whether the test compound stabilized the tight junction complexes.

8. The method of claim 7, wherein the junction proteins are selected from the group consisting of integral membrane proteins, cytoplasmic proteins, and proteins associated with tight junctions.

9. The method of claim 7, wherein the junction proteins are selected from the group consisting of occludin, claudin, zonula occludens (ZO)-1, -2, -3, catenins, cingulin and p130.

10. The method of claim 7, wherein the compound that stabilizes the tight junction complexes is an anti-permeability and/or an anti-angiogenic compound.

11. A method of screening for compounds that affect vascular permeability, comprising:
a) assaying endothelial cells on a permeable substrate;
b) contacting the assay with a test compound;
c) treating the assay with a marker and a permeability-inducing agent; and
d) measuring the rate of diffusion of the marker compare to control.

12. A method for assessing bioeffectiveness of an antiangiogenic compound in a patient being treated with said compound comprising:
a) measuring a level of a protein in a bodily fluid of the patient before treating the patient with the antiangiogenic compound;
b) treating the patient with the antiangiogenic compound;
c) measuring the level of the protein in the bodily fluid of the patient subsequent to treating the patient with the antiangiogenic compound, wherein a decreased level of protein in the bodily fluid indicates that the compound is bioeffective.

13. The method of claim 12, wherein the bodily fluid is urine, peripheral blood or plasma.

14. An article of manufacture comprising packaging material and a pharmaceutical agent contained within said packaging material, wherein said packaging material comprises a label which indicates said pharmaceutical may be administered, for a sufficient term at an effective dose, for treating and/or preventing a disease associated with vascular permeability, wherein said pharmaceutical agent comprises a compound selected from the group consisting of endostatin, thrombospondin, angiostatin, tumstatin, arrestin, recombinant EPO, and polymer conjugated TNP-470.

15. The article of manufacture of claim 14, wherein the disease associated with vascular permeability is selected from the group consisting of non-proliferative diabetic retinopathy, diabetic nephropathy, nephrotic syndrome, macular degeneration, psoriasis, pulmonary hypertension, side effects of treatment with interleukins, burn edema, tumor edema, brain tumor edema, IL-2 therapy-associated edema, and other edema-associated diseases.

16. A method of decreasing or inhibiting vascular hyperpermeability in an individual in need thereof, comprising administering to said individual an effective amount of compound selected from the group consisting of a taxane and derivatives thereof; alpha, beta or gamma interferon; IL-12; matrix metalloproteinases inhibitors; a Cox-2 inhibitor; a PDGFR inhibitor; a EGFR1 inhibitor and a Bisphosphonate.

17. The method of claim 16, wherein the vascular permeability is the result of a disease selected from the group consisting of non-proliferative diabetic retinopathy, diabetic nephropathy, nephrotic syndrome, pulmonary hypertension, allergic reactions associated with edema, lymphedema, cerebral edema, brain tumor edema, burn edema, tumor edema, reperfusion syndromes, and IL-2 therapy-associated edema.
